(19) United States
(12) Patent Application Publication
(10) Pub. No.: US 2011/0152218 A1
(43) Pub. Date: Jun. 23, 2011

(54) CRYSTALLINE PHOSPHOLIPID, METHOD FOR ITS PRODUCTION AND USE IN TREATING DAMAGED TISSUE

(76) Inventor: Derek Woodcock, Surrey (GB)

(21) Appl. No.: 12/920,974

(22) PCT Filed: Mar. 4, 2009
(86) PCT No.: PCT/GB2009/000597

§ 371 Date: Dec. 13, 2010

(30) Foreign Application Priority Data
Mar. 4, 2008 (GB) 0803969.5
Dec. 3, 2008 (GB) 0822093.1

(51) Int. Cl. A61K 31/685; C07F 9/09; C07F 9/12; A61P 17/02; A61P 43/00; B29C 35/16

(52) U.S. Cl. 514/77; 558/166; 558/177; 558/169; 514/119; 514/148; 264/28

(57) ABSTRACT

The present invention provides phospholipid in a crystalline form, a method for its preparation, compositions comprising it and its use in the treatment of damaged tissue.

The invention provides an improved phospholipid, its therapeutic use and a method for its production.

A problem with formulating a phospholipid is that it can be difficult to form an aqueous dispersions of a phospholipid without the formation of large agglomerations or lumps which make it difficult to administer the phospholipid and which make the phospholipid therapeutically inactive. Accordingly, there is a further need to provide a phospholipid which may be administered as an aqueous dispersion without the formation of large agglomerations. There is also a need to find a way of sterilising a phospholipid without reducing its activity and/or without using organic solvents.

A way of ameliorating these problems has been sought.

According to the invention there is provided a first phospholipid in a crystalline form.

The advantages of the invention include that the phospholipid according to the invention may be dispersed in an aqueous solvent without the formation of lumps or agglomerations. Further advantages of the phospholipid according to the invention include that it has improved therapeutic activity and that it contains substantially no organic solvents. The improved therapeutic activity is evident from the data in Examples 2 to 6 of the present application. This data shows the effectiveness of a phospholipid according to the invention in promoting wound healing.

In some aspects, the phospholipid according to the invention may be freeze dried. In some aspects, the phospholipid according to the invention may be sterilised. In some aspects, the phospholipid may be particulate. In some embodiments, the phospholipid may be in the form of a respirable particle. A respirable particle may have a mass median aerodynamic diameter suitable for pulmonary administration, for example the mass median aerodynamic diameter of a respirable particle may be less than 10 μm. In some embodiments, a respirable particle is a micronized particle. The phospholipid according to the invention generally has a crystalline appearance such as that of fine crystals of sugar. The phospholipid according to the invention may be in a form such that when dispersed at room temperature in a polar solvent (for example water), especially an isotonic polar solvent (e.g. M199), the dispersion formed comprises discrete crystals or particles. The discrete crystals or particles may be visible discrete crystals or particles when viewed at a magnification of at least 5×, especially 50×. The phospholipid according to the invention may be in a form such that when dispersed at 37° C. in a polar solvent (for example water), especially an isotonic polar solvent (e.g. M199), the dispersion formed comprises a fine micellar dispersion of the phospholipid according to the invention which has substantially no visible agglomerations of micelles. The fine micellar dispersion may have a substantially even particle size distribution. The fine micellar dispersion may be visible when viewed at a magnification of at least 5×, especially 50×.

In some aspects, the phospholipid according to the invention may have the following general formula:

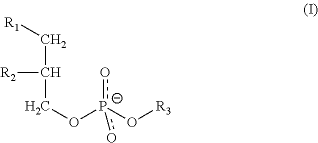

wherein R₁ and R₂ each independently represents a hydrogen atom or a fatty acid acyl residue (in some aspects, R₁ and R₂ each independently represents a straight or branched chain saturated or unsaturated acyl group having from 14, preferably from 16 to 22, preferably to 20 carbon atoms), and R₃ represents a hydrogen atom or a choline, glycerol, ethanolamine, serine or inositol group wherein R₁ and R₂ cannot both represent a hydrogen atom.

In some aspects, a fatty acid acyl residue independently represented by R₁ or R₂ may be a saturated radical such as palmitoyl C16:0 and stearoyl C18:0 and/or an unsaturated radical such as oleoyls C18:1 and C18:2. In some aspects, R₁ and R₂ may not represent a hydrogen atom such that the phospholipid is a diacyl. In some aspects, R₁ and R₂ may represent identical saturated or unsaturated acyl radicals, especially dipalmitoyl and distearoyl. In some aspects, the phospholipid may be a mixture of such phospholipids, in particular mixtures in which dipalmitoyl is the major diacyl component.

The phospholipid is optionally either animal-derived or plant-derived or synthetically produced. An artificial phospholipid is generally understood to be a phospholipid that does not occur in nature; preferably it is a synthetic phospholipid free from risk of including animal-derived protein. The phospholipid may be an animal-derived or synthetic phospholipid as defined above.

The phospholipid is preferably used in a method of medical treatment as the sole active ingredient. Accordingly, the pharmaceutical composition according to the invention preferably only comprises the phospholipid as a therapeutic agent. The phospholipid is preferably substantially free from cholesterol or a tri-glyceride. The phospholipid is also preferably substantially free from a cryoprotectant.

The phospholipid is preferably a mixture of a diacyl phosphatidyl choline and a phosphatidyl glycerol. The phosphatidyl glycerol is advantageously a diacyl phosphatidyl glycerol. The acyl groups of the phosphatidyl glycerol, which may be the same or different, are advantageously each fatty acid acyl groups which may have from 14 to 22 carbon atoms. In practice, the phosphatidyl glycerol component may be a mixture of phosphatidyl glycerols containing different acyl groups. It is preferred for at least a proportion of the fatty acid acyl groups of the phosphatidyl glycerol to be an unsaturated fatty acid acyl residue, for example, a mono- or di-unsaturated C18 or C20 fatty acid acyl residue.

Preferred acyl substituents in the phosphatidyl glycerol component are palmitoyl, oleoyl, linoleoyl, linolenoyl, myristoyl and arachidonoyl. The phospholipid preferably comprises dipalmitoyl phosphatidyl choline and phosphatidyl glycerol.

The phospholipid is preferably a mixture of DPPC and PG at a weight ratio of from 1:9 to 9:1, preferably from 6:4 to 8:2, more preferably about 7:3. DPPC can be prepared synthetically by acylation of glyceryl phosphoryl choline using the method of Baer & Bachrea, Can. J. of Biochem. Physiol 1959, 37, page 953 and is available commercially from Sigma (London) Ltd. PG may be prepared from egg phosphatidyl-choline by the methods of Comfurions et al, Biochem. Biophys Acta 1977, 488, pages 36 to 42; and Dawson, Biochem J. 1967, 102, pages 205 to 210, or from other phosphatidyl cholines, such as soy lecithin.

The present invention surprisingly solves the problem with making DPPC wholly therapeutically active which arises because the temperature at which it becomes surface active is greater than body temperature. The temperature of the phase transition from solid phase to liquid phase at a liquid (e.g. aqueous) boundary for DPPC is 41° C. which is greater than body temperature which is 37° C. There have been two main approaches to obtaining DPPC in a therapeutically active form. These are to use an animal-derived phospholipid which contains DPPC as a mixture of other phospholipids, optionally with proteins, fatty acids and/or cholesterol or to use a synthetic phospholipid which is a non-naturally occurring mixture of phospholipids and other compounds.

A animal-derived phospholipid may be obtained in the usual way by mincing of or lavage from mammalian lungs, such as porcine or bovine lungs. Examples of animal-derived phospholipids include Curosurf™ (Chiesi Farmaceutici) which is produced from minced pig lungs and consists of 99% phospholipids and 1% surfactant proteins; Alveofact™ (Dr. Karl Thomae, Ltd., Germany) which is a compound obtained from bovine lung lavage and contains 90% phospholipids, about 1% proteins, 3% cholesterol, 0.5% free fatty acids, and other components, including triglycerides; Survanta™ (Abbott, Ltd., Germany) which is prepared by lipid extraction of minced bovine lungs and contains approximately 84% phospholipids, 1% proteins, and 6% free fatty acids; BLES (BLES Biochemicals, Canada) which is produced by a bovine lung lavage; or Infasurf™ (Forest Labs) also known as calfactant which is produced by bovine calf lung lavage and contains 35 mg/ml phospholipids which are 26 mg/ml phosphatidyl choline (PC), 26 mg/ml dipalmitoylphosphatidylcholine (DPPC), 0.65 mg/ml protein and 0.26 mg/ml a hydrophobic peptide.

A synthetic phospholipid is preferably a diacyl phosphatidyl choline (DAPC) such as DPPC, dioleyl phosphatidyl choline (DOPC) or distearyl phosphatidyl choline (DSPC), phosphatidylglycerol (PG), PC, phosphatidylethanolamine (PE), phosphatidylserine (PS), phosphatidylinositol (PI), phosphatidic acid, and/or a lysophospholipid.

Examples of commercial synthetic phospholipid products include: Surfaxin™ (Discovery Labs) which is also known as lucinactant contains 26 molar parts of DPPC, 8 molar parts of POPG, 5 molar parts of PA and 1 part of KL-4; Lung Surfactant Factor LSF (Altana) which is also known as lusupultide contains recombinant SP-C, DPPC, PG and PA; Exosurf™ (GSK, Germany) which is composed of DPPC (˜84%), cetyl alcohol, and tyloxapol; or pumactant (Britannia Pharmaceuticals) which is composed of a mixture of DPPC and PG at a weight ratio of 7:3.

The problem with the known approaches to make DPPC therapeutically active is that they are expensive; the concentration of DPPC is reduced and they may include additives which have other effects. The present invention provides a solution to this problem by making DPPC therapeutically active by providing it in a form which is readily dissoluble in water.

The phospholipid is preferably a phospholipid or a mixture of phospholipids which has a melting or transition temperature which is about the same as or below body temperature (which is the temperature of the human or animal body to be treated). The relevant transition temperature is the temperature at which the phospholipid will spread on a liquid surface and/or become surface active. Such a mixture of phospholipids preferably contains a spreading phospholipid which has a melting temperature which is about the same as or below body temperature such as PG, PE, PS, or PI.

According to the invention, there is further provided a process of preparing a phospholipid according to the invention which method comprises:

dispersing a phospholipid in a polar solvent;

homogenising the phospholipid dispersion;

filtering the homogenised phospholipid to sterilise it; and

freeze drying the filtered phospholipid dispersion.

It was surprising that in the process of the invention, it was possible to filter the homogenised phospholipid. It was expected that the dispersion of the phospholipid in the polar solvent would form agglomerations which would block the pores of the filter. The process according to the invention provides a procedure for putting DPPC into a form where it is therapeutically more active. Thus the process provides an alternative means for providing a therapeutically active form of DPPC without further dilution of DPPC or the introduction of additives which might have an associated risk of side effects.

The filtering step preferably uses a 0.5 μm filter, more preferably a 0.2 μm filter. In some aspects, the sterilisation step uses pressure filtration.

The polar solvent used in the dispersing step of the process according to the invention is generally a polar solvent in which the phospholipid forms a liposome dispersion. In some aspects, the polar solvent may be water. Some phospholipids, such as pumactant, are temperature and/or water sensitive. Accordingly, it is surprising that the process of the invention is effective in producing a therapeutically active phospholipid.

In the homogenising step, the size of the liposomes in the liposome dispersion is reduced. In some aspects, the homogenising step uses a homogeniser. The homogeniser may be an ultrasonic, pressure or mechanical homogeniser.

The freeze drying step is preferably carried out without using a cryoprotectant. A cryoprotectant is a substance which is used to protect a material from freezing damage, for example due to the formation of ice crystals. Examples of cryoprotectants which are generally used include a sugar molecule such as glucose, sucrose, mannitol or trehalose.

The dispersing, homogenising and/or the sterilising step is carried out at a temperature above the transition temperature of the phospholipid. The relevant transition temperature of the phospholipid is the temperature at which the phospholipid changes phase from a solid phase to a liquid phase in the presence of a liquid, i.e. the temperature at which the phospholipid will spread on a liquid surface and/or become surface active.

According to the invention, there is also provided a process of preparing a phospholipid according to the invention which method comprises:

dispersing a phospholipid in a polar solvent;

homogenising the phospholipid dispersion;

freeze drying the phospholipid dispersion;

micronising the freeze dried phospholipid. The method optionally includes a filtering step.

According to the invention there is also provided a second phospholipid obtainable by the process of the invention.

According to the invention there is further provided a pharmaceutical composition which comprises a first or second phospholipid according to the invention in association with a pharmaceutically acceptable diluent.

The pharmaceutical composition according to the invention comprises a pharmaceutically acceptable diluent or vehicle. Any compatible diluent or vehicle may be used. In some aspects, the diluent is an isotonic polar solvent, for example saline, phosphate buffered saline (PBS), ringer's lactate, water for injection, M199 and/or Hartmann's solution. In some aspects, the diluent may be supplemented. In some aspects, the diluent may be substantially free from glycerol. In some aspects, the diluent may be substantially free from sodium hydroxide. In some aspects, the diluent may comprise a buffer, for example Tris and/or lactate.

In some aspects, the pharmaceutically acceptable diluent used in the invention comprises a surface active agent. A surface active agent is useful because it enables a phospholipid having a melting temperature above body temperature to be used in the composition. More preferably the surface active agent is a pharmaceutically acceptable surfactant or hydrophobic protein. Examples of such agents include: KL-4 which is 21 amino acid synthetic peptide; tyloxapol which is a nonionic surfactant; cetyl alcohol (or hexadecanol); or cholesteryl palmitate. A further suitable diluent is a protein, especially a protein which improves absorption such as apoprotein B.

According to the invention there is also provided a kit for preparing a pharmaceutical composition according to the invention which kit has at least two parts wherein a first part comprises a first or second phospholipid according to the invention and a second part comprises a pharmaceutically acceptable diluent.

In some aspects, each part of the kit according to the invention is provided in a receptacle, for example in a pouch or vial.

According to the invention there is provided a first or second phospholipid according to the invention or a pharmaceutical composition according to the invention for use in the treatment of damaged tissue.

According to the invention there is also provided use of a first or second phospholipid according to the invention or of a pharmaceutical composition according to the invention in the manufacture of a medicament for use in the treatment of damaged tissue.

According to the invention, there is further provided a method of treating damaged tissue which method comprises applying to a human or animal patient in need of such treatment a therapeutically effective amount of a first or second phospholipid according to the invention. The first or second phospholipid is preferably in the form of a pharmaceutical composition according to the invention.

The phospholipid is preferably applied in an amount of from 1, preferably from 10 μg, more preferably from 50 μg to 1000 mg, preferably to 800 mg, more preferably to 300 mg. The amount of phospholipid applied may depend upon the area or volume of the damaged tissue or on the body mass of the patient.

In some aspects, the treatment of damaged tissue in the invention comprises facilitating re-epithelialisation, for example of a keratinocyte, synovial lining cell of the tendon sheath or other epithelial cell. A further repair process which might be facilitated by the present invention is the alteration of the phenotype or function of an intrinsic or migrating cell (e.g. a fibroblast cell). In other words, the phospholipid is preferably used to treat damaged tissue by facilitating re-epithelialisation and/or alteration of the phenotype/function of an intrinsic cell type. By re-epithelialisation is meant re-growth of epithelial or other surface cells.

The damaged tissue to be treated by the invention may be external or internal tissue. The damaged tissue may be skin, organ tissue, connective tissue (especially load-bearing connective tissue (e.g. tendon tissue and/or a tendon sheath) or a membrane (e.g. an epithelial or connective tissue membrane). In some aspects, damaged tissue to be treated by the invention is an opening or abrasion on a surface of a human or animal body. The surface of a human or animal body to be treated is optionally either an internal or external surface. The tissue may be damaged as a result of physical trauma or may be a result of a chronic inflammatory process. Examples of physical trauma include, for example, hurt or injury caused by a burn, accidental or surgical incision, violent or disruptive action. The internal physical trauma treated by the invention is internal accidental physical trauma caused by injury as a result of an accident or intentional action such as surgery. External physical trauma to be treated by the present invention includes external accidental physical trauma as well as external surgical physical trauma caused by surgery such as for example trauma caused by the removal of skin for a skin graft. The damaged tissue is optionally a site on the surface of a human or animal body where the condition of the surface is accidentally degraded such as a burn, opening or abrasion. The damage to the tissue may have been exacerbated by inflammation. An animal to be treated by the present invention may be a mammal.

The phospholipid or pharmaceutical composition according to the invention is preferably applied topically to the wound.

In some aspects, the treatment of damaged tissue in the present invention comprises the treatment of tissue damaged in a surgical procedure to prevent a surgical adhesion. The data in Example 12 shows that treatment with pumactant resulted in a statistically significant reduction in histology/presence of adhesions.

The invention is illustrated with reference to the following Figures of drawings which are not intended to limit the scope of the claims and in which:

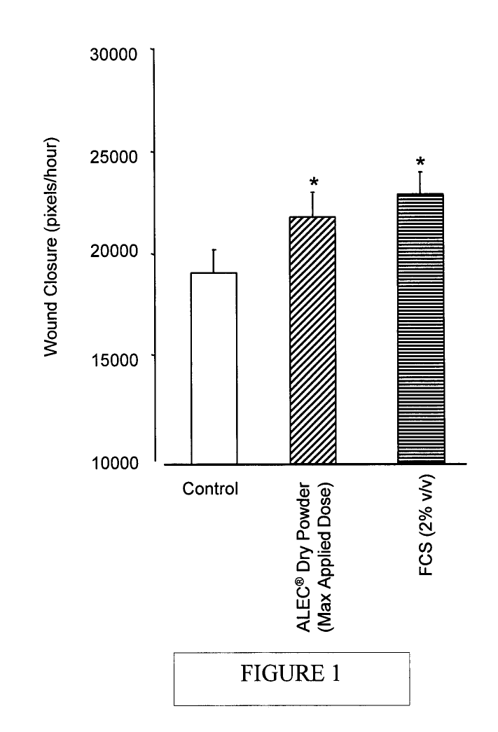

FIG. 1 shows a bar chart having a scale of the rate of re-mesothelialisation following scratch wounding (pixels/hour) on the y-axis wherein the first bar from the left has no cross-hatching and represents the wound closure rate for the control, the second bar from the left has diagonal cross-hatching and represents the wound closure rate for ALEC® and the third bar from the left has horizontal cross-hatching and represents the wound closure rate for 2% v/v of foetal calf serum;

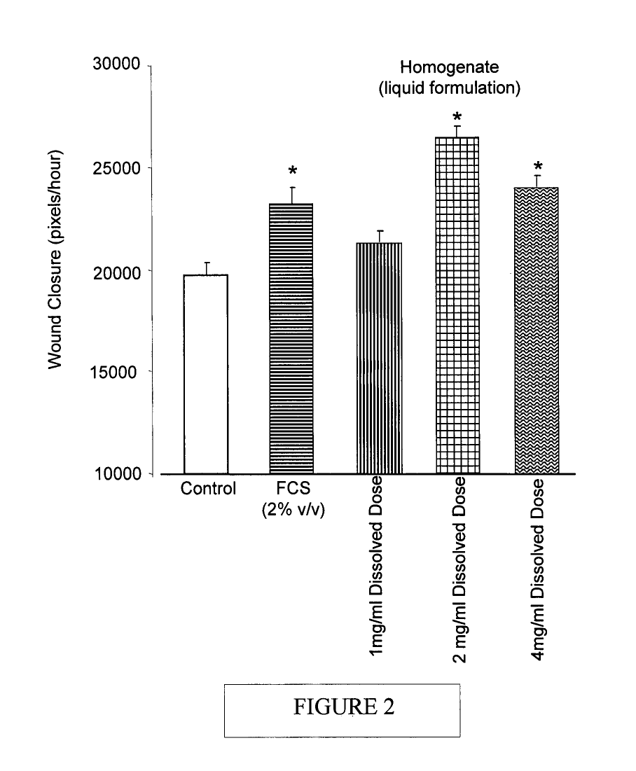

FIG. 2 shows a bar chart having a scale of the rate of re-mesothelialisation following scratch wounding (pixels/hour) on the y-axis wherein the first bar from the left has no cross-hatching and represents the wound closure rate for the control, the second bar from the left has horizontal cross-hatching and represents the wound closure rate for 2% v/v of foetal calf serum, the third bar from the left has vertical cross-hatching and represents the wound closure rate for a 1 mg/ml dose of a phospholipid prepared according to Example 1, the fourth bar from the left has checked cross-hatching and represents the wound closure rate for a 2 mg/ml dose of a phospholipid prepared according to Example 1 and the fifth bar from the left has wavy horizontal cross-hatching and represents the wound closure rate for a 4 mg/ml dose of a phospholipid prepared according to Example 1;

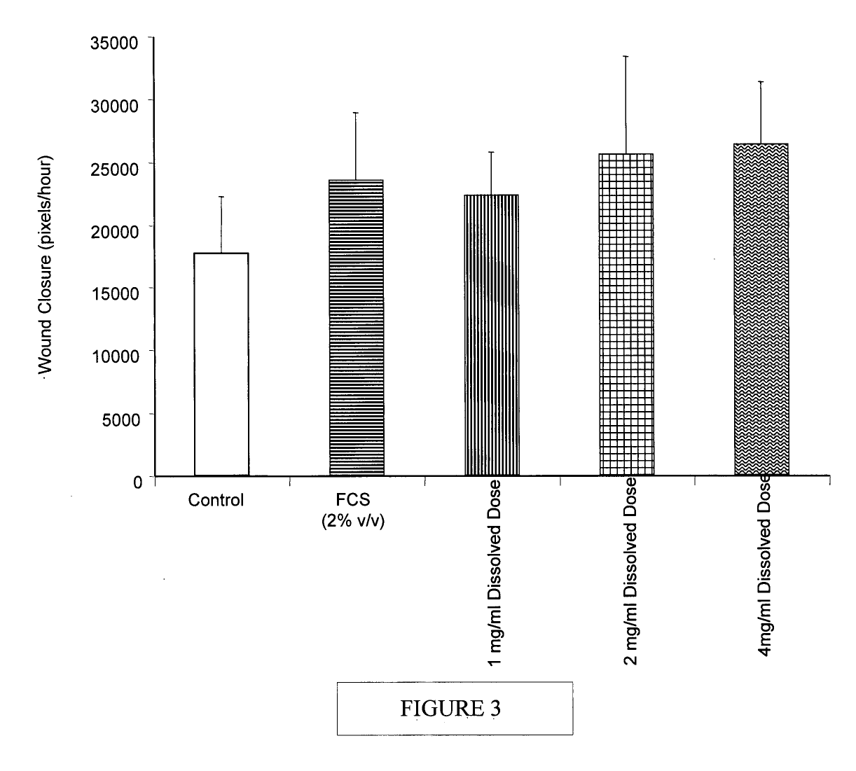

FIG. 3 shows a bar chart having a scale on the y-axis of the rate of NHEK-A repair following scratch wounding (pixels/hour) wherein the first bar from the left has no cross-hatching and represents the wound closure rate for the control, the second bar from the left has horizontal cross-hatching and represents the wound closure rate for 2% v/v of foetal calf serum, the third bar from the left has vertical cross-hatching and represents the wound closure rate for a 1 mg/ml dose of a phospholipid prepared according to Example 1, the fourth bar from the left has checked cross-hatching and represents the wound closure rate for a 2 mg/ml dose of a phospholipid prepared according to Example 1 and the fifth bar from the left has wavy horizontal cross-hatching and represents the wound closure rate for a 4 mg/ml dose of a phospholipid prepared according to Example 1;

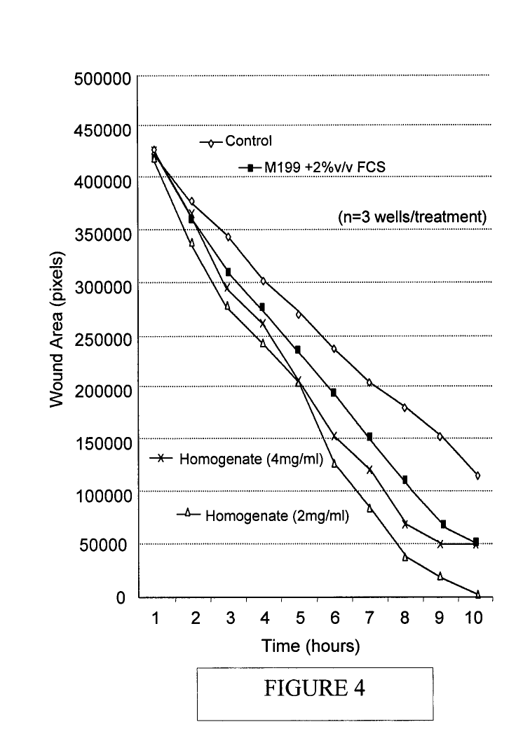

FIG. 4 shows a graph having a scale of wound area (measured in pixels) of HPMC cells on its y-axis and time (hours) on its x-axis wherein data points for a wound treated with a 4 mg/ml dose of a phospholipid prepared according to Example 1 are shown by “X”, data points for a wound treated with a 2 mg/ml dose of a phospholipid prepared according to Example 1 are shown by “▴”, the control data points are shown by “♦”, and the data points for a wound treated with M199 and 2% v/v foetal calf serum are shown by “▪”;

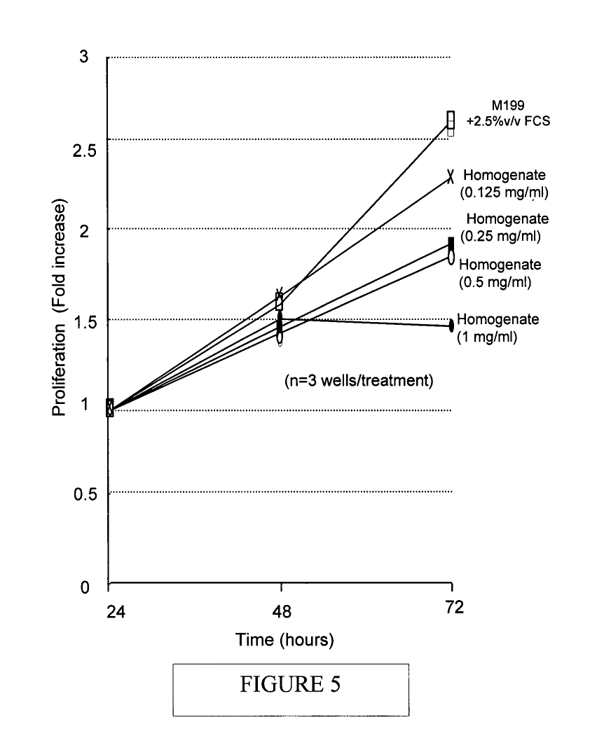

FIG. 5 shows a graph having a scale of proliferation (fold increase) of HPMC cells on its y-axis and time (hours) on its x-axis wherein data points for cells treated with a 0.125 mg/ml dose of a phospholipid prepared according to Example 1 are shown by “X”; data points for cells treated with a 0.25 mg/ml dose of a phospholipid prepared according to Example 1 are shown by “▪”; data points for cells treated with a 0.5 mg/ml dose of a phospholipid prepared according to Example 1 are shown by “0”; data points for cells treated with 1 mg/ml dose of a phospholipid prepared according to Example 1 are shown by “•”; and, data points for cells treated with M199 and 2.5% v/v foetal calf serum are shown by “□”;

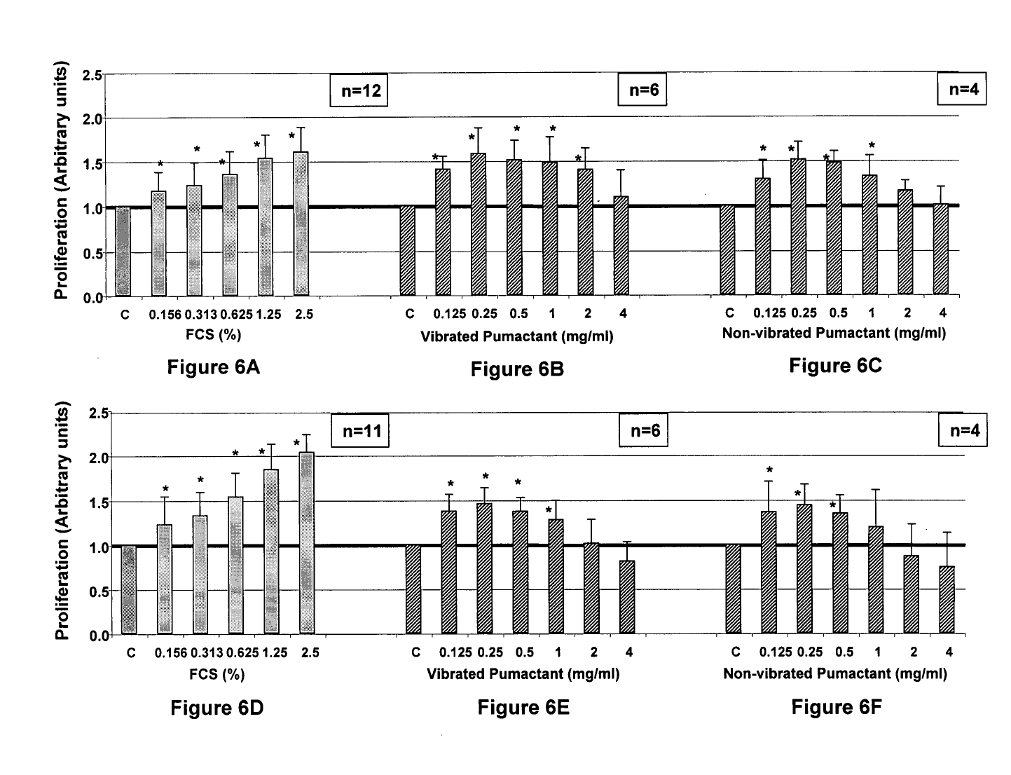

FIGS. 6A-6F show bar charts of the results of HPMC proliferation assays: FIG. 6A shows the results after 48 hours for cells treated with the indicated v/v % amounts of FCS; FIG. 6B shows the results after 48 hours for cells treated with the indicated mg/ml amounts of vibrated phospholipid prepared as described in Example 1; FIG. 6C shows the results after 48 hours for cells treated with the indicated mg/ml amounts of non-vibrated phospholipid prepared as described in Example 1; FIG. 6D shows the results after 72 hours for cells treated with the indicated v/v % amounts of FCS; FIG. 6E shows the results after 72 hours for cells treated with the indicated mg/ml amounts of vibrated phospholipid prepared as described in Example 1; FIG. 6F shows the results after 72 hours for cells treated with the indicated mg/ml amounts of non-vibrated phospholipid prepared as described in Example 1; wherein the y-axis has a scale of proliferation (measured in arbitrary units relative to the control), error bars indicate standard deviations, n represents the number of wells tested and *p<0.05 compared to negative control (C) which were cells stimulated with tissue culture medium (M199) without serum;

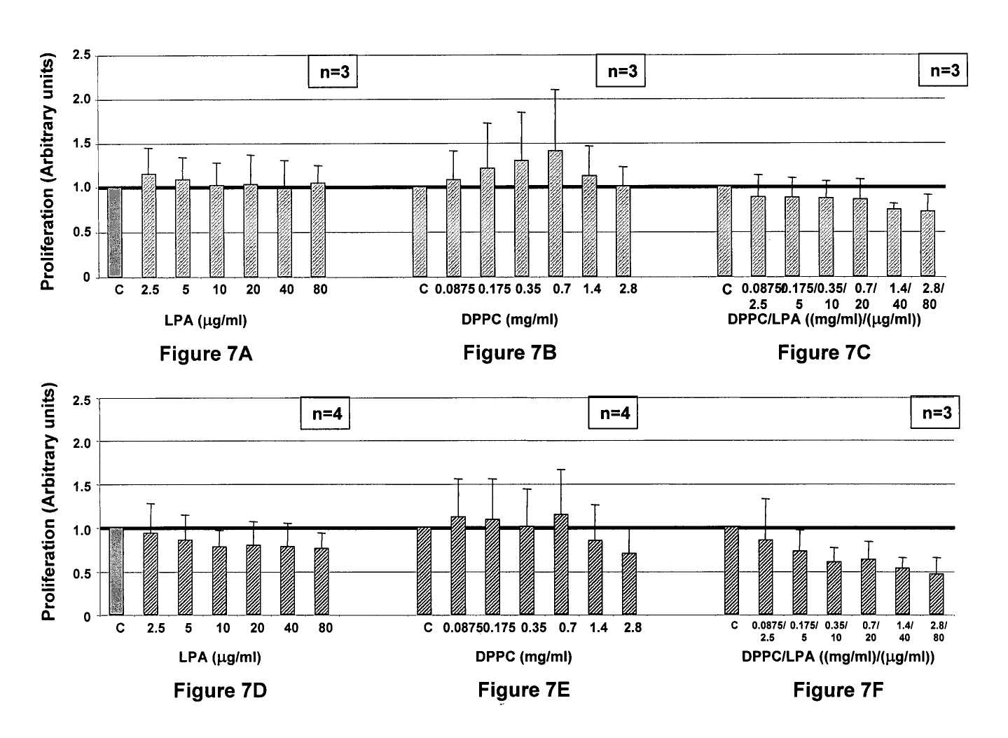

FIGS. 7A-7F show bar charts of the results of HPMC proliferation assays: FIG. 7A shows the results after 48 hours for cells treated with the indicated μg/ml amounts of lysophosphatidic acid (LPA); FIG. 7B shows the results after 48 hours for cells treated with the indicated mg/ml amounts of DPPC; FIG. 7C shows the results after 48 hours for cells treated with the indicated (mg/ml)/(μg/ml) amounts of DPPC/LPA; FIG. 7D shows the results after 72 hours for cells treated with the indicated μg/ml amounts of lysophosphatidic acid (LPA); FIG. 7E shows the results after 72 hours for cells treated with the indicated mg/ml amounts of DPPC; FIG. 7F shows the results after 72 hours for cells treated with the indicated (mg/ml)/(μg/ml) amounts of DPPC/LPA; wherein the y-axis has a scale of proliferation (measured in arbitrary units relative to the control), error bars indicate standard deviations, n represents the number of wells tested and the negative control (C) is for cells stimulated with tissue culture medium (M199) without serum;

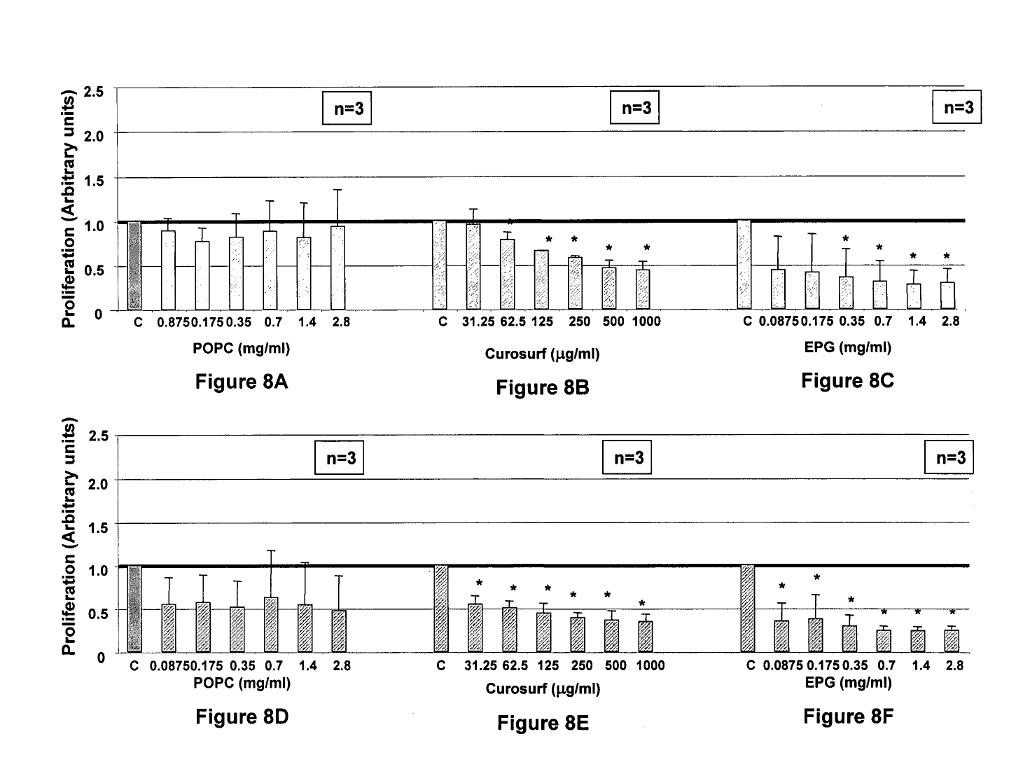

FIGS. 8A-8F show bar charts of the results of HPMC proliferation assays: FIG. 8A shows the results after 48 hours for cells treated with the indicated mg/ml amounts of POPC; FIG. 8B shows the results after 48 hours for cells treated with the indicated μg/ml amounts of Curosurf (Trade Mark); FIG. 8C shows the results after 48 hours for cells treated with the indicated mg/ml amounts of egg-PG (EPG); FIG. 8D shows the results after 72 hours for cells treated with the indicated mg/ml amounts of POPC; FIG. 8E shows the results after 72 hours for cells treated with the indicated μg/ml amounts of Curosurf (Trade Mark); FIG. 8F shows the results after 72 hours for cells treated with the indicated mg/ml amounts of egg-PG; wherein the y-axis has a scale of proliferation (measured in arbitrary units relative to the control), error bars indicate standard deviations, n represents the number of wells tested and *p<0.05 compared to negative control (C) which were cells stimulated with tissue culture medium (M199) without serum;

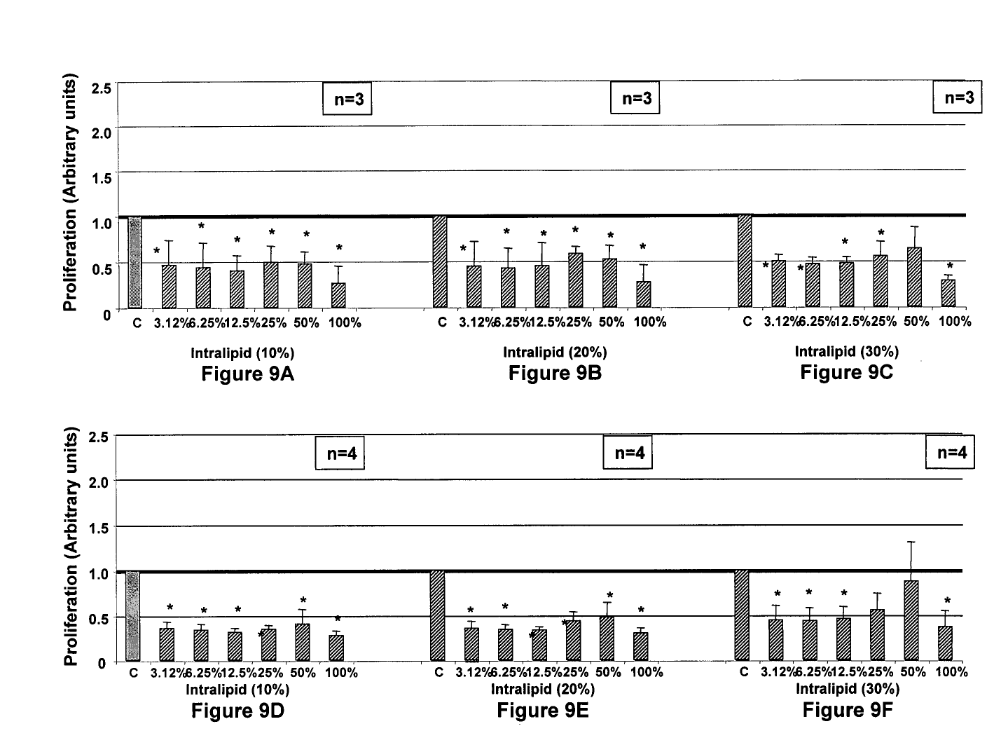

FIGS. 9A-9F show bar charts of the results of HPMC proliferation assays: FIG. 9A shows the results after 48 hours for cells treated with the indicated v/v percentages of 10% v/v of a fat emulsion of Intralipid (Trade Mark) in serum free tissue culture medium (M199); FIG. 9B shows the results after 48 hours for cells treated with the indicated v/v percentages of 20% v/v of a fat emulsion of Intralipid in serum free tissue culture medium (M199); FIG. 9C shows the results after 48 hours for cells treated with the indicated v/v percentages of 30% v/v of a fat emulsion of Intralipid in serum free tissue culture medium (M199); FIG. 9D shows the results after 72 hours for cells treated with the indicated v/v percentages of 10% v/v of a fat emulsion of Intralipid (Trade Mark) in serum free tissue culture medium (M199); FIG. 9E shows the results after 72 hours for cells treated with the indicated v/v percentages of 20% v/v of a fat emulsion of Intralipid in serum free tissue culture medium (M199); FIG. 9F shows the results after 72 hours for cells treated with the indicated v/v percentages of 30% v/v of a fat emulsion of Intralipid in serum free tissue culture medium (M199); wherein the y-axis has a scale of proliferation (measured in arbitrary units relative to the control), error bars indicate standard deviations, n represents the number of wells tested and *p<0.05 compared to negative control (C) which were cells stimulated with tissue culture medium (M199) without serum;

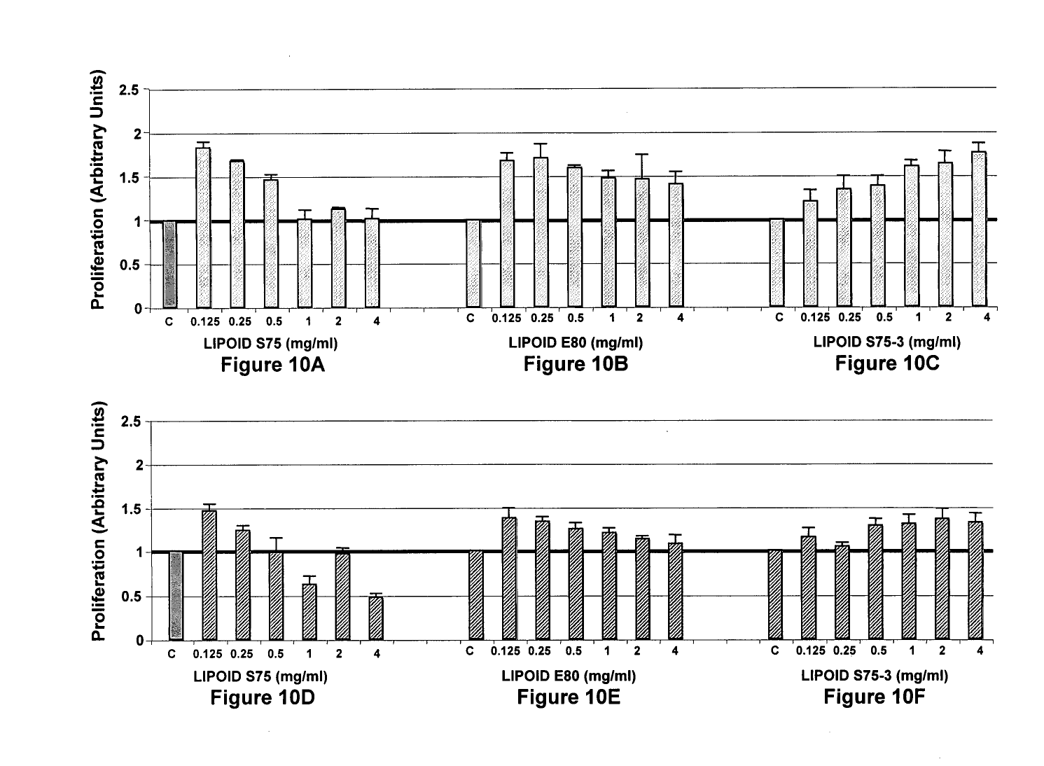

FIGS. 10A-10F show bar charts of the results of HPMC proliferation assays: FIG. 10A shows the results after 48 hours for cells treated with the indicated mg/ml amounts of Lipoid phospholipid product S75 in serum free tissue culture medium (M199); FIG. 10B shows the results after 48 hours for cells treated with the indicated mg/ml amounts of Lipoid phospholipid product E80 in serum free tissue culture medium (M199); FIG. 10C shows the results after 48 hours for cells treated with the indicated mg/ml amounts of Lipoid phospholipid product S75-3 in serum free tissue culture medium (M199); FIG. 10D shows the results after 72 hours for cells treated with the indicated mg/ml amounts of Lipoid phospholipid product S75 in serum free tissue culture medium (M199); FIG. 10E shows the results after 72 hours for cells treated with the indicated mg/ml amounts of Lipoid phospholipid product E80 in serum free tissue culture medium (M199); FIG. 10F shows the results after 72 hours for cells treated with the indicated mg/ml amounts of Lipoid phospholipid product S75-3 in serum free tissue culture medium (M199); wherein the y-axis has a scale of proliferation (measured in arbitrary units relative to the control), error bars indicate standard deviations, n represents the number of wells tested and the negative control (C) is for cells which were stimulated with tissue culture medium (M199) without serum;

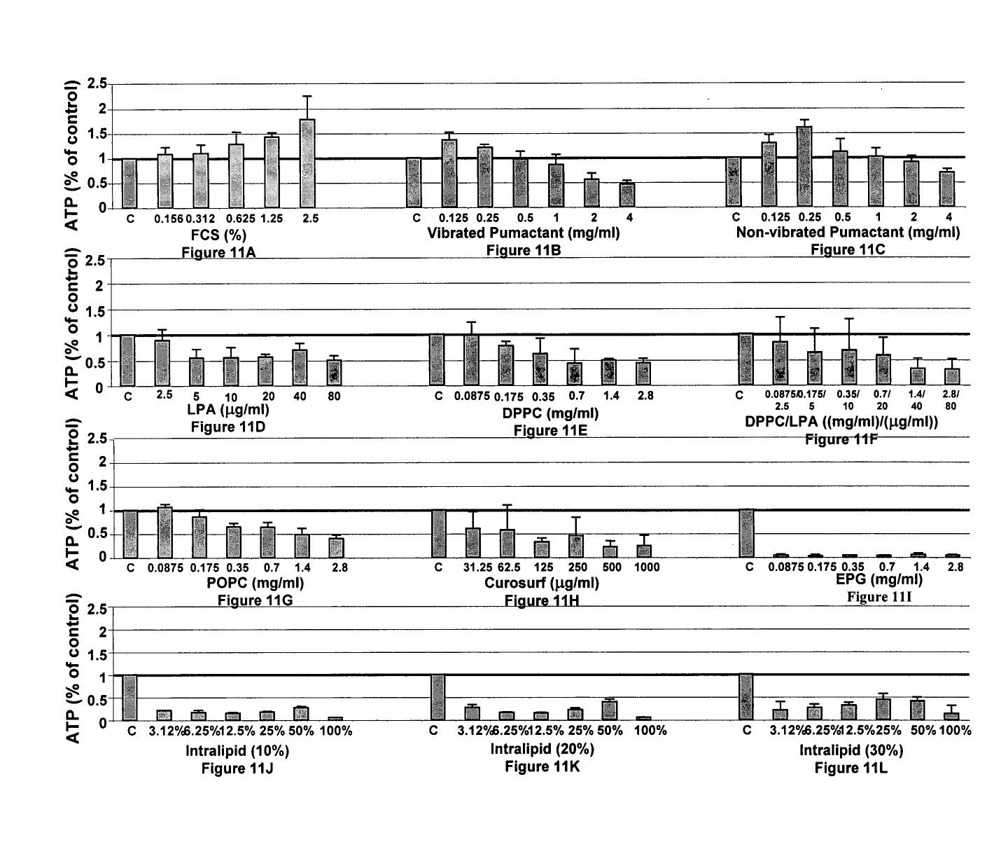

FIGS. 11A-11L show bar charts of the results of an HPMC ATP assays after 72 hours for a single well of cells treated (FIG. 11A) with the indicated v/v % amounts of FCS; (FIG. 11B) with the indicated mg/ml amounts of vibrated phospholipid prepared as described in Example 1; (FIG. 11C) with the indicated mg/ml amounts of non-vibrated phospholipid prepared as described in Example 1; (FIG. 11D) with the indicated μg/ml amounts of LPA; (FIG. 11E) with the indicated mg/ml amounts of DPPC; (FIG. 11F) with the indicated (mg/ml)/(μg/ml) amounts of DPPC/LPA; (FIG. 11G) with the indicated mg/ml amounts of POPC; (FIG. 11H) with the indicated μg/ml amounts of Curosurf™; (FIG. 11I) with the indicated mg/ml amounts of egg-PG; (FIG. 11J) with the indicated v/v percentages of 10% v/v of a fat emulsion of Intralipid™ in serum free tissue culture medium (M199); (FIG. 11K) with the indicated v/v percentages of 20% v/v of a fat emulsion of Intralipid™ in serum free tissue culture medium (M199); (FIG. 11L) with the indicated v/v percentages of 30% v/v of a fat emulsion of Intralipid™ in serum free tissue culture medium (M199); wherein the y-axis shows an ATP value as a percentage of the control value;

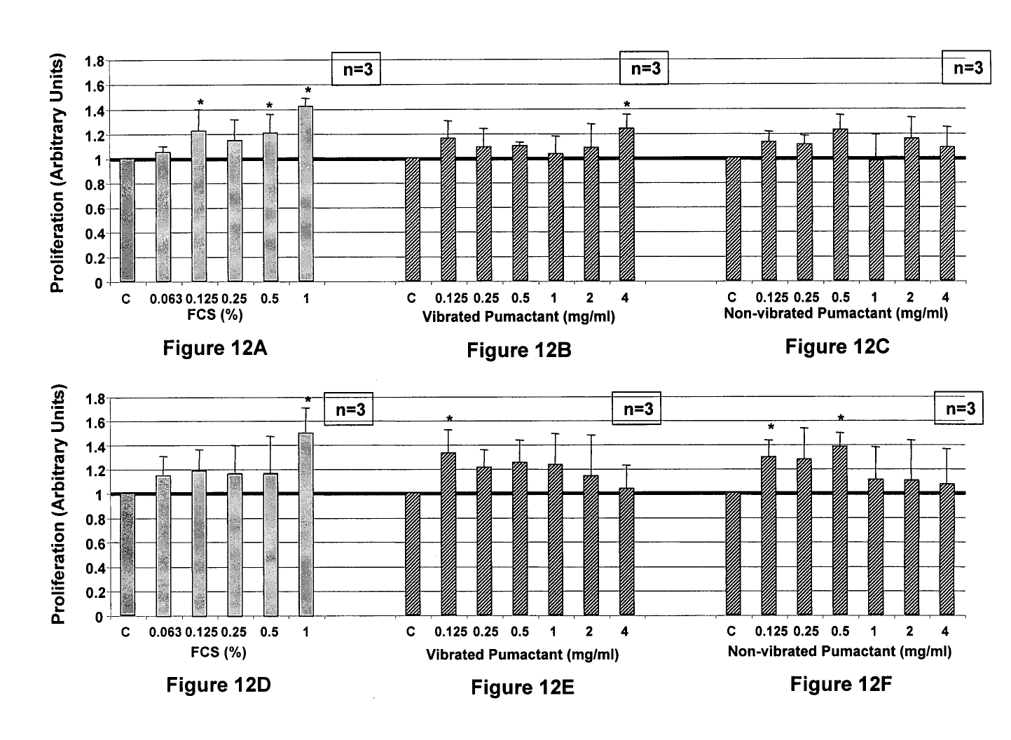

FIGS. 12A-12F show bar charts of the results of NEKa proliferation assays: FIG. 12A shows the results after 48 hours for cells treated with the indicated v/v % amounts of FCS; FIG. 12B shows the results after 48 hours for cells treated with the indicated mg/ml amounts of vibrated phospholipid prepared as described in Example 1; FIG. 12C shows the results after 48 hours for cells treated with the indicated mg/ml amounts of non-vibrated phospholipid prepared as described in Example 1; FIG. 12D shows the results after 72 hours for cells treated with the indicated v/v % amounts of FCS; FIG. 12E shows the results after 72 hours for cells treated with the indicated mg/ml amounts of vibrated phospholipid prepared as described in Example 1; FIG. 12F shows the results after 72 hours for cells treated with the indicated mg/ml amounts of non-vibrated phospholipid prepared as described in Example 1; wherein the y-axis has a scale of proliferation (measured in arbitrary units relative to the control), error bars indicate standard deviations;

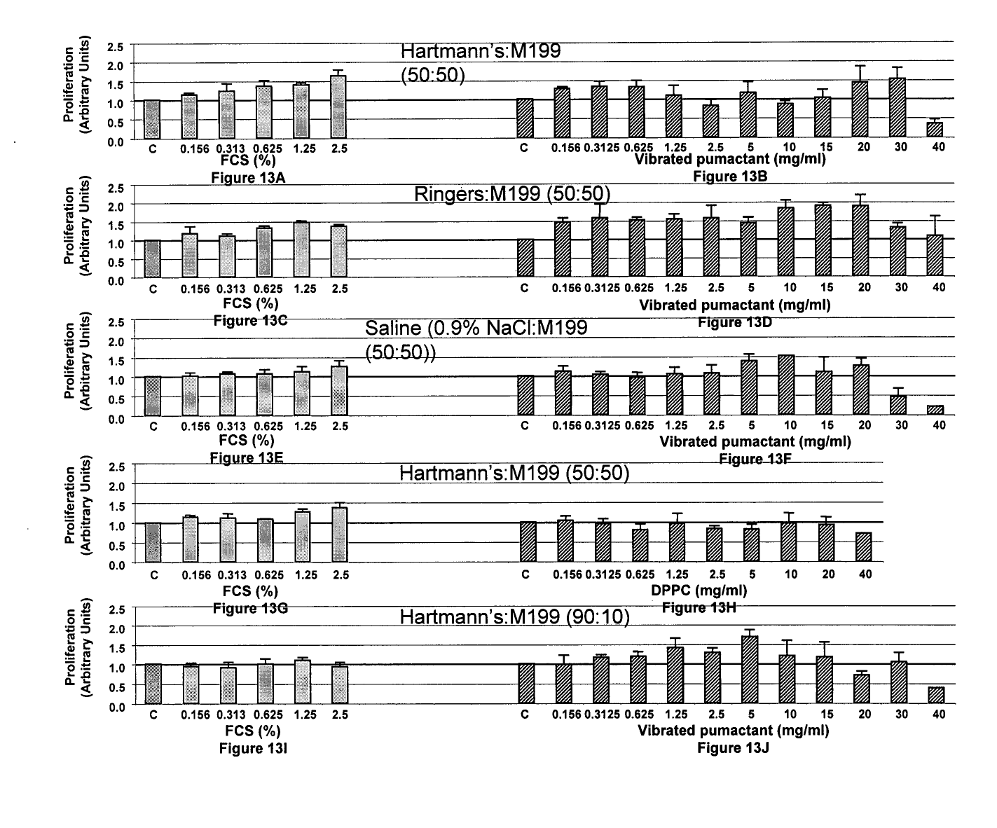

FIGS. 13A-13J show bar charts of the results of HPMC proliferation assays after 48 hours for a single well of cells treated: (FIGS. 13A and 13B) with 2% v/v FCS and the indicated amounts in mg/ml of vibrated phospholipid prepared as described in Example 1 using a 50:50 v/v mixture of Hartmann's solution and M199 as the medium; (FIGS. 13C and 13D) with 2% v/v FCS and the indicated amounts in mg/ml of vibrated phospholipid prepared as described in Example 1 using a 50:50 v/v mixture of Ringers solution and M199 as the medium; (FIGS. 13E and 13F) with 2% v/v FCS and the indicated amounts in mg/ml of vibrated phospholipid prepared as described in Example 1 using a 50:50 v/v mixture of a saline solution (containing 0.9% w/v sodium chloride/water) and M199 as the medium; (FIGS. 13G and 13H) with 2% v/v FCS and the indicated amounts in mg/ml of DPPC using a 50:50 v/v mixture of Hartmann's solution and M199 as the medium; and (FIGS. 131 and 13J) with 2% v/v FCS and the indicated amounts in mg/ml of vibrated phospholipid prepared as described in Example 1 using a 90:10 v/v mixture of Hartmanns solution and M199 as the medium; wherein the y-axis has a scale of proliferation (measured in arbitrary units relative to the control), error bars indicate standard deviations;

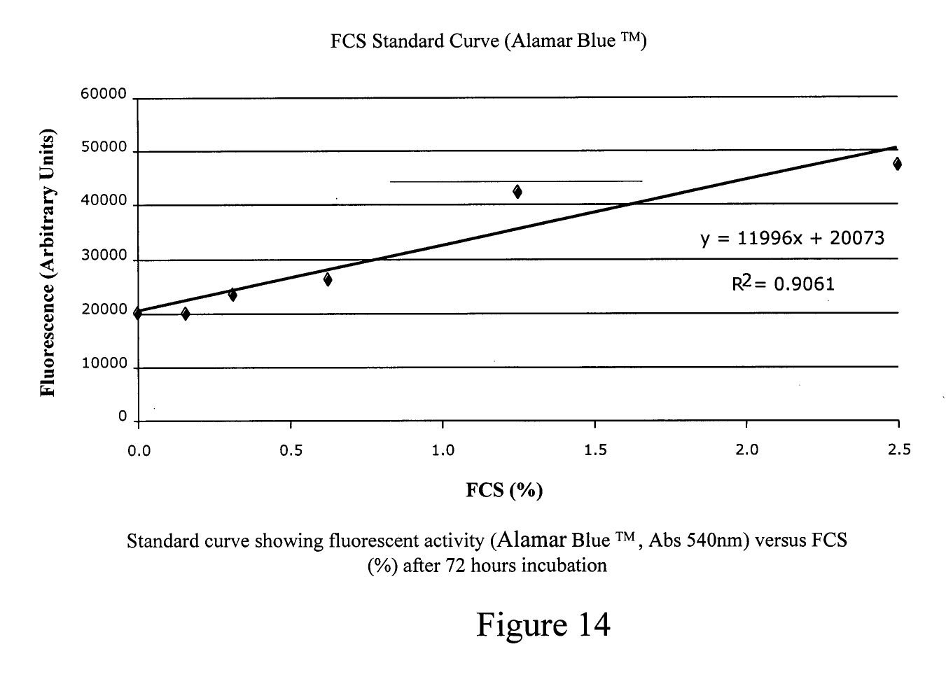

FIG. 14 shows a standard curve showing fluorescent activity (Alamar Blue™, Abs 540 nm) versus FCS (%) after 72 hours incubation;

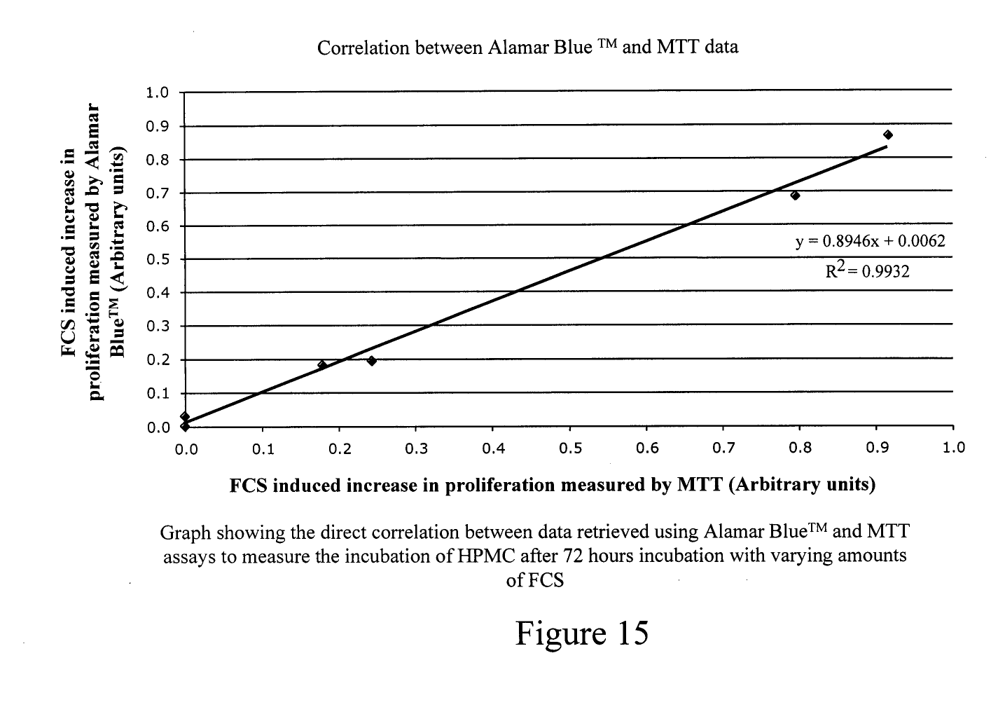

FIG. 15 shows a graph showing the direct correlation between data retrieved using the Alamar Blue™ and MTT assays to measure the incubation of HPMC after 72 hours incubation with varying amounts of FCS;

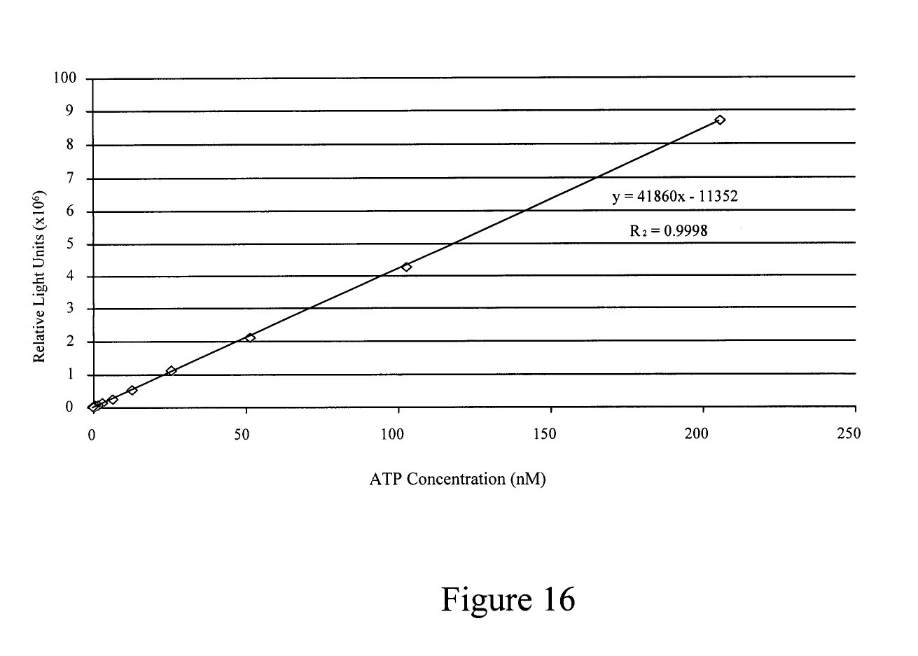

FIG. 16 shows an ATP standard curve prepared using the ATP standard curve procedure set out in Example 4 and shows relative light units (×10⁶) versus ATP concentration (nM);

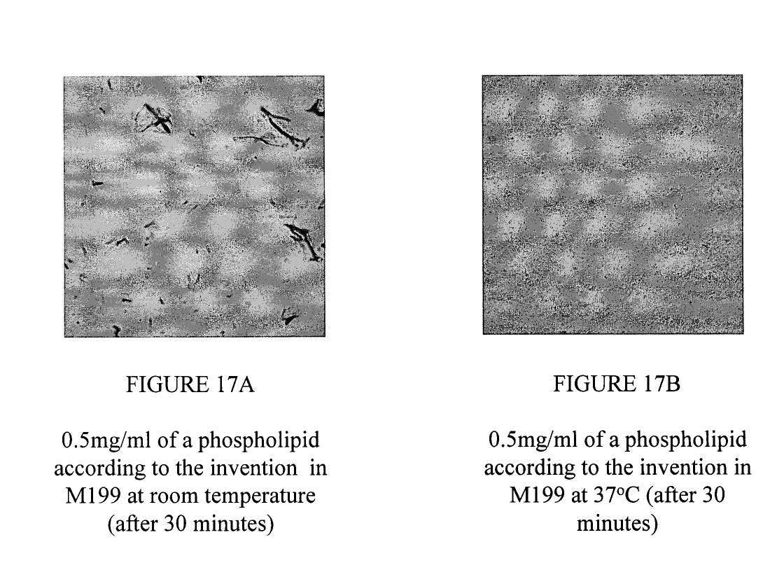

FIGS. 17A and 17B show micrographs of a dispersion of 0.5 mg/ml of a phospholipid prepared as described in Example 1 in M199 at room temperature wherein the micrograph shown in FIG. 17A was taken after the dispersion had been at room temperature for 30 minutes and the micrograph shown in FIG. 17B was taken after the dispersion had been at 37° C. for 30 minutes; Images were taken using an Axiovert 100M inverted microscope with a 5× objective (Carl Zeiss; Oberkochen, Germany) such that the final magnification in the images shown is 50×;

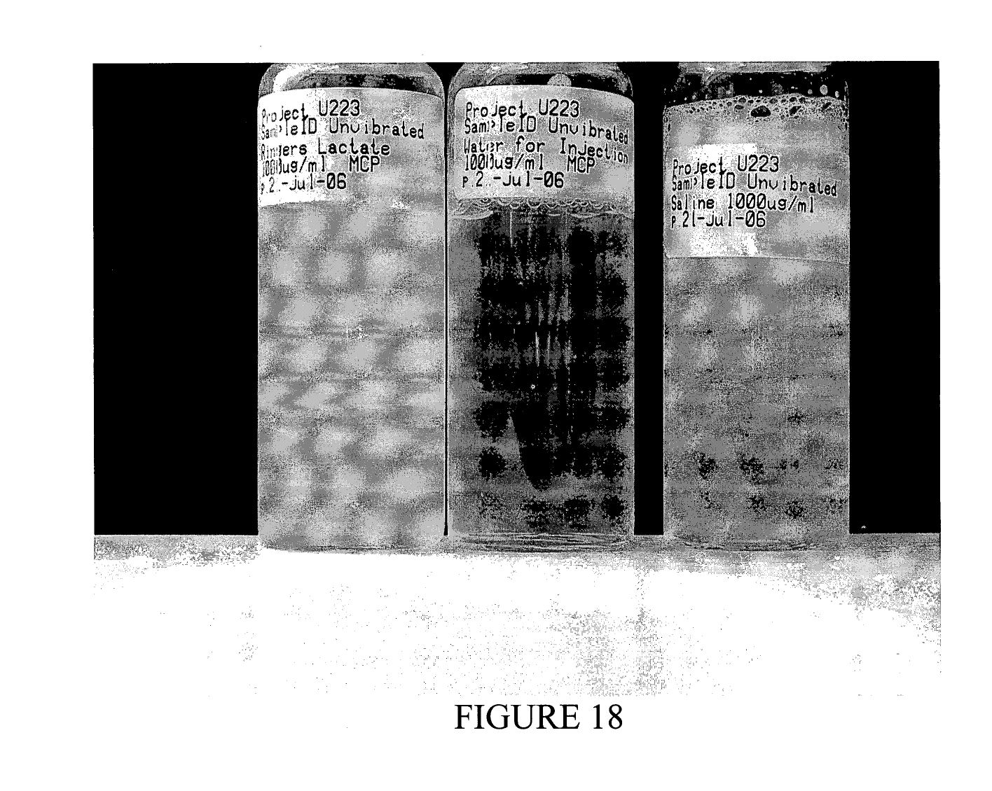

FIG. 18 shows a photograph of three vials containing dispersions of 1 mg/ml of an unvibrated phospholipid prepared as described in Example 1 wherein the medium used to form the dispersion is Ringers lactate in the left hand vial, water for injection in the middle vial and saline in the right hand vial.

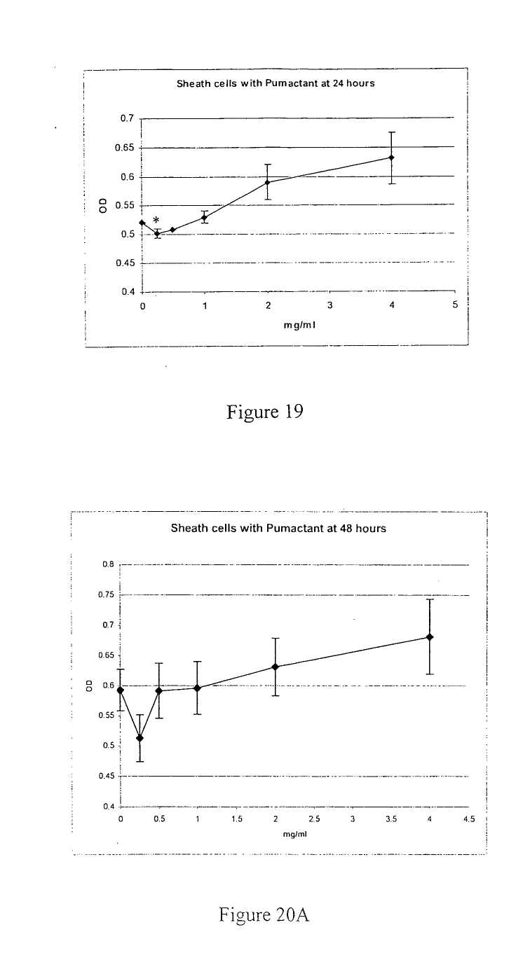

FIG. 19 shows graphs representing the average (n=3) and SD for Sheath cells at 24 hours with Pumactant. A statistical decrease was demonstrated at 0.25 mg/ml of Pumactant (*p=0.04). Sheath cells cultured for 24 hours in various percentages of fetal calf serum or various concentrations of TGF-β1 showed no statistical changes in cell number (data not shown);

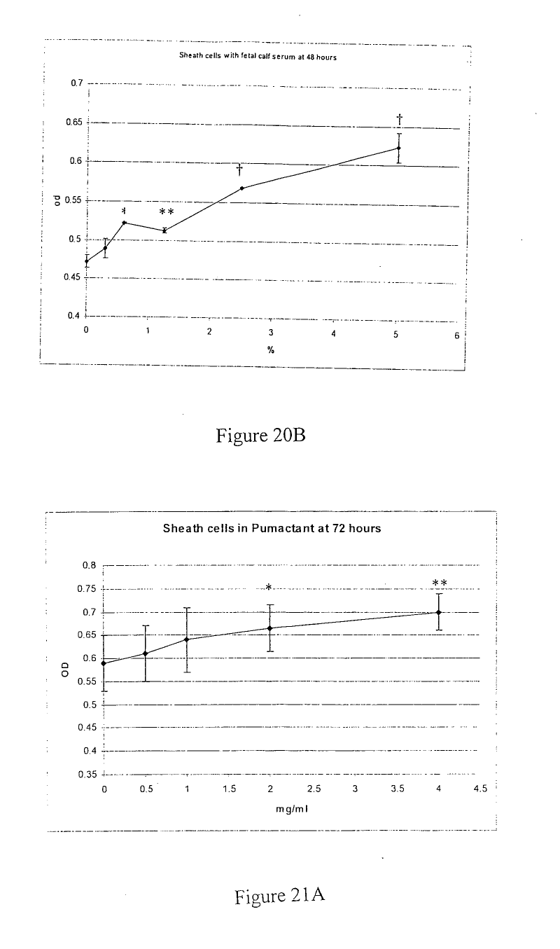

FIGS. 20A and 20B show graphs representing the average (n=7) and SEM for Sheath cells at 48 hours with Pumactant (FIG. 20A), Fetal calf serum (n=3; FIG. 20B). Sheath cells in 0.6% fetal calf serum showed a statistical significant increase in proliferation (*p=0.025) and this was also seen at 1.25% (**p=0.03), 2.5% (†p=0.003) and 5% (‡p=0.01). Cells cultured in TGF-β1 showed no statistical significance for any concentration (data not shown);

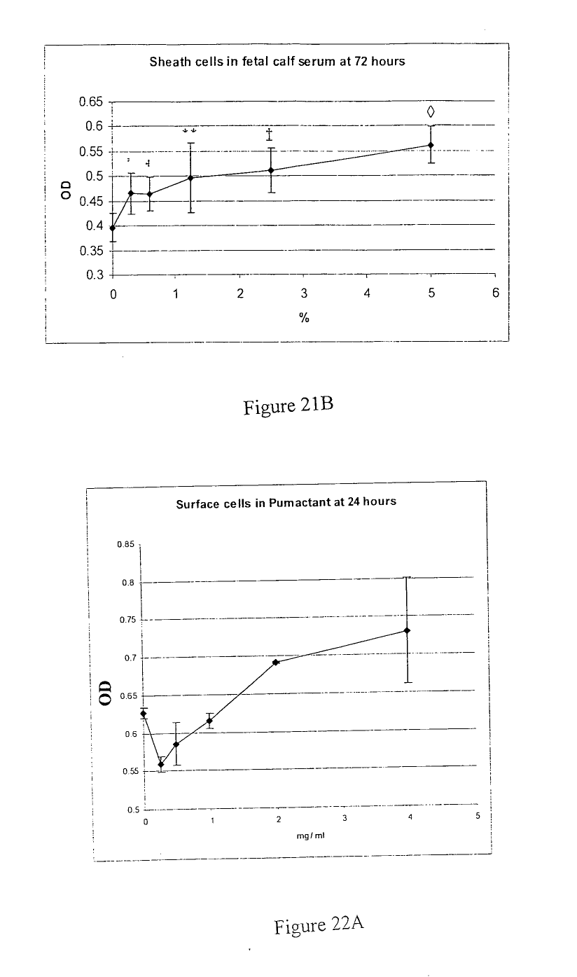

FIGS. 21A and 21B show graphs representing the average (n=7) and SEM for Sheath cells at 72 hours with Pumactant (FIG. 21A). Pumactant showed a statistical significant increase at 4 mg/ml (**p=0.022) and 2 mg/ml (*p=0.018). Cells grown in fetal calf serum showed a statistically significant increase compared to those grown in serum free media (*p=0.03, †p=0.002, **p=0.04, ‡p=0.004, ⋄p=0.005; FIG. 21B). Tendon sheath cells in 0.5 ng/ml of TGF-β1 showed a statistical significant increase (*p=0.022);

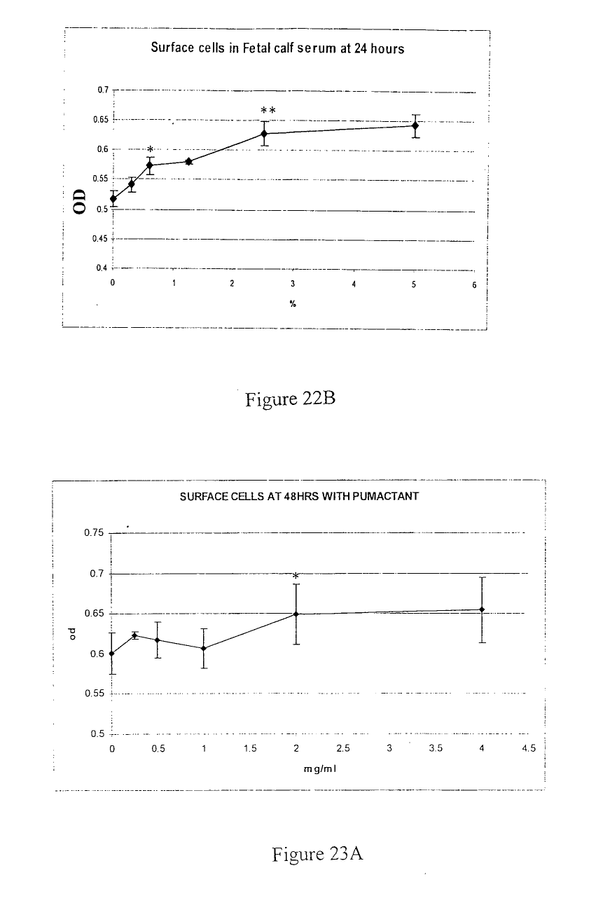

FIGS. 22A and 22B show graphs representing the average (n=3) and SEM for Surface cells at 24 hours with pumactant (FIG. 22A), Fetal calf serum (FIG. 22B). The only statistical difference increase was for surface derived tendon cells cultured in 0.6% (*p=0.027) and 2.5% (**p=0.001) fetal calf serum compared to those cultured in serum free media;

FIGS. 23A and 23B show graphs representing the average (n=6) and SEM for Surface cells at 48 hours with pumactant (FIG. 23A), and Fetal calf serum (FIG. 23B). The only statistical differences were an increase for pumactant at 2 mg/ml (*p=0.047) compared to those grown in minimal media and an increase in cells grown in 0.3% (*p=0.009), 1.25% (**p=0.005), 2.5% (†p=0.017) 5% (‡p=0.01) fetal calf serum compared to those in serum free media. Those cultured with various concentrations of TGF-β1 showed no statistical significance (data not shown);

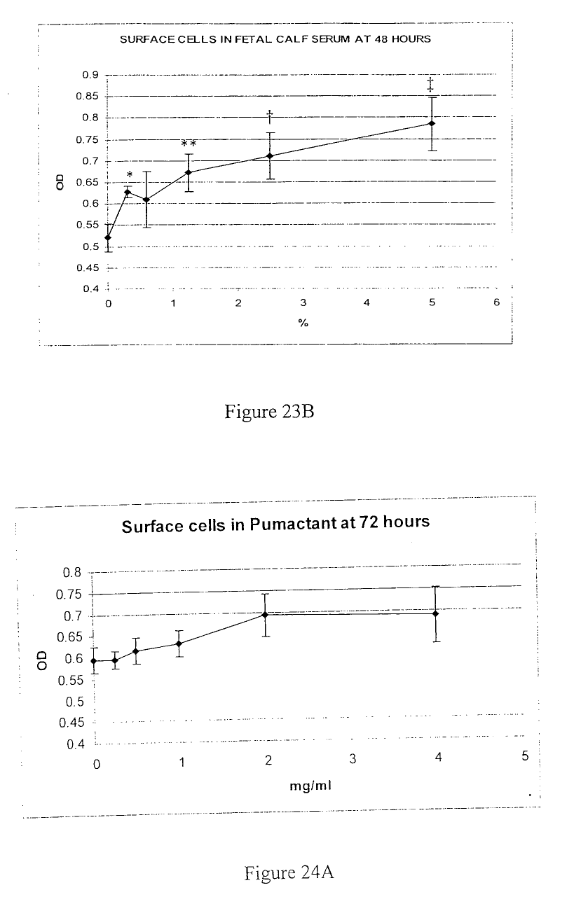

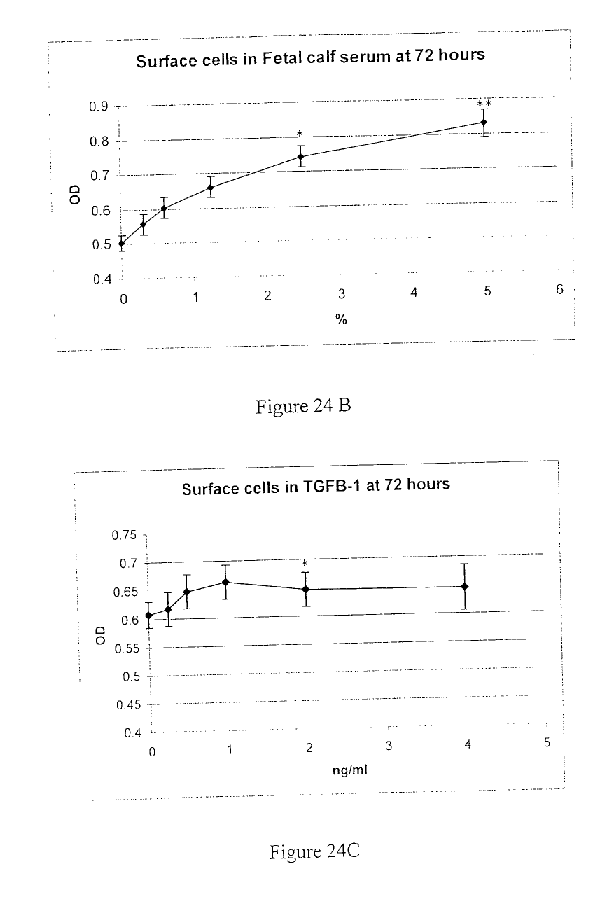

FIGS. 24A, 24B and 24C show graphs representing the average and SEM for Surface cells at 72 hours with pumactant (n=6; FIG. 24A), Fetal calf serum (n=3; FIG. 24B) and TGF-β1 (n=3; FIG. 24C). Cells cultured in pumactant showed no statistical significance for any concentration. The only statistical differences were an increase for surface cells grown in fetal calf serum at 2.5% (*p=0.03) and 5% (**p=0.044) compared to those in serum free media and an increase was identified at 2 ng/ml (*p=0.003) compared to those solely in minimal media;

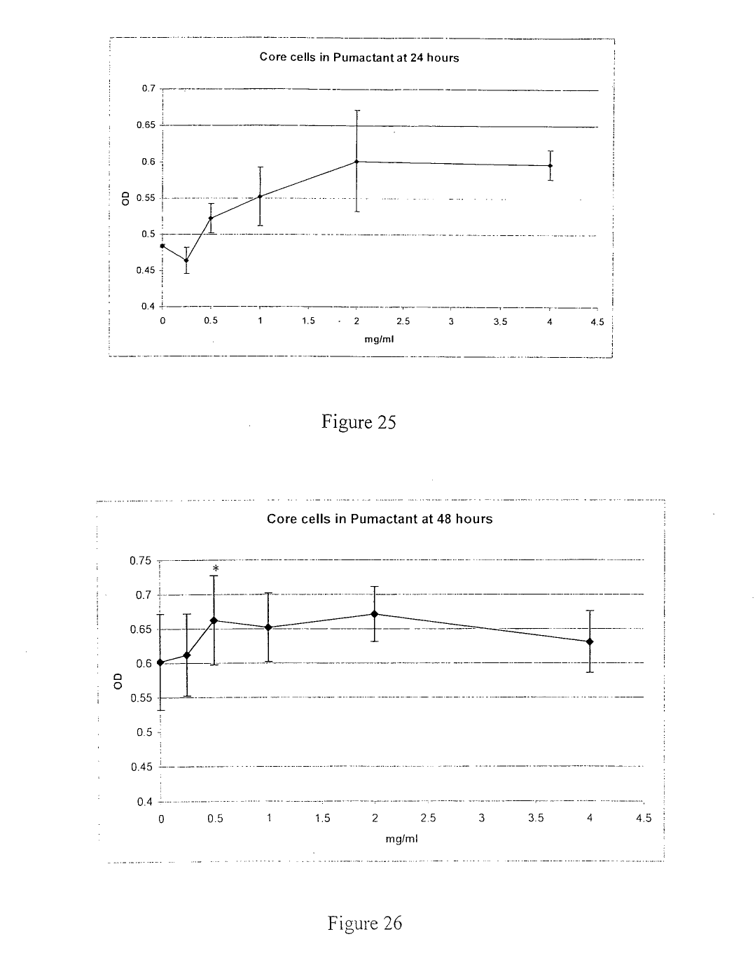

FIG. 25 shows a graph representing the average and SEM for Core cells at 24 hours with pumactant (n=3) There was no statistical differences for any of the treatments;

FIG. 26 shows a graph representing the average and SEM for Core cells at 48 hours with pumactant (n=5). Pumactant showed a statistical increase at 0.5 mg/ml compared to core cells grown in minimal media (*p=0.049). Core cells cultured in various concentrations in FCS or TGF-β1 showed no statistical significance (data not shown);

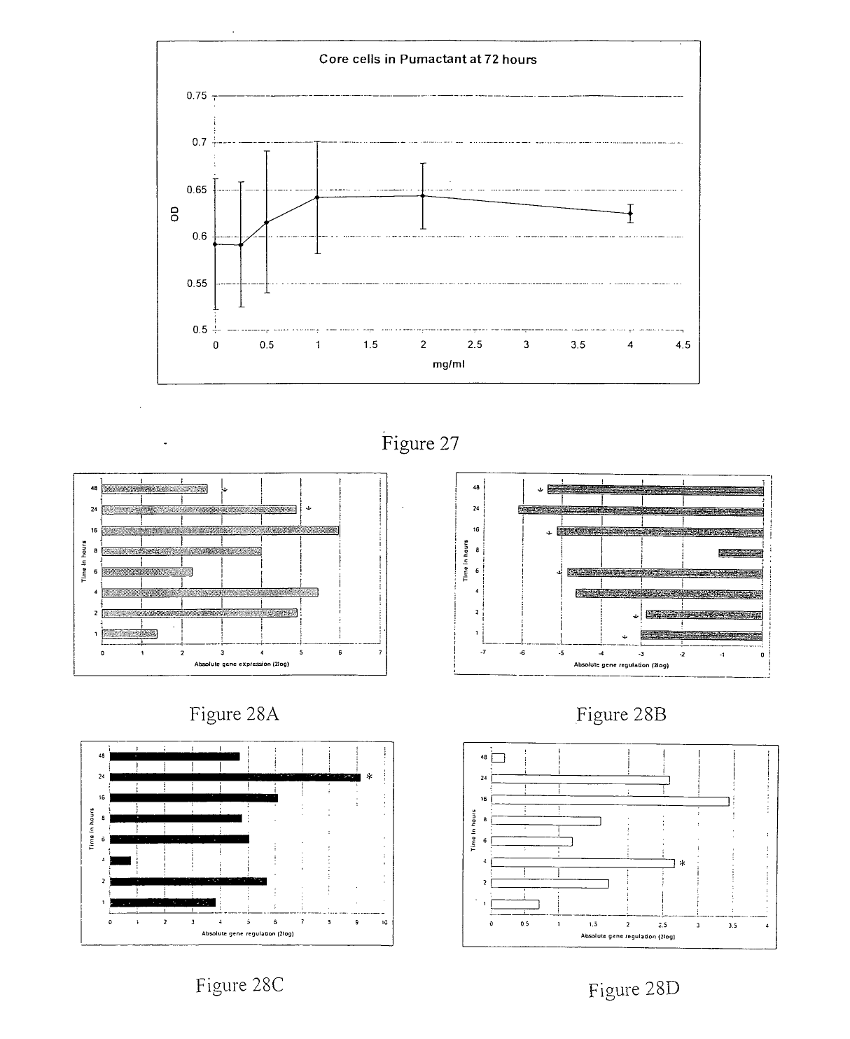

FIG. 27 shows a graph representing the average and SEM for Core cells at 72 hours with Pumactant (n=6). There was no statistical significance with any concentration. Various concentrations of fetal calf serum or TGF-β1 showed no statistical significance (data not shown);

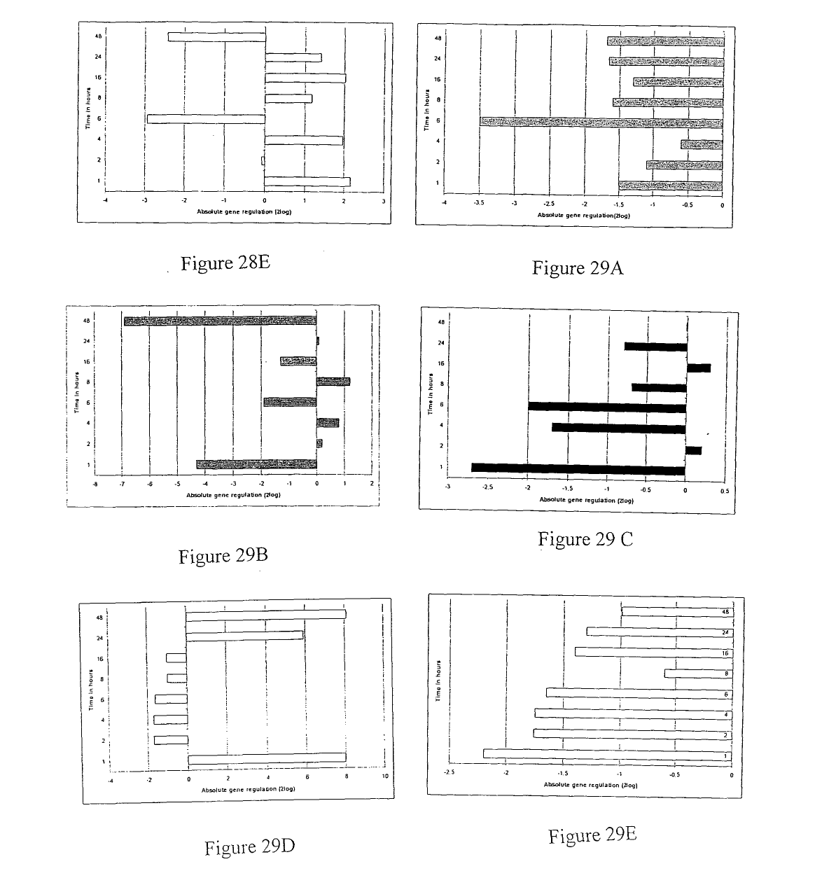

FIGS. 28A to 28E show TGF-β1 effect on gene transcription on surface derived cells (n=3). Graphs represent the absolute gene regulation. FIG. 28A shows the surface-derived cells response to TGF-β1 for collagen type I (Grey bars) which showed a statistically significant up regulation at 24 hours (*p=0.025) and 48 hours (*p=0.04). FIG. 28B represents the graph for Collagen type III (stripe bars) which showed a statistically significant down regulation at 1 hrs (*p=0.036), 2 hrs (*p=0.001), 6 hrs (*p=0.038), 16 hrs and 48 hrs (*p=0.001), FIG. 28C represents the graph for fibronectin gene expression (black bars) which showed a statistically significant up regulation at 24 hours (*p=0.03). FIG. 28D represents the graph for PAI-1 (spots), which showed a statistically significant up regulation at 4 hrs (*p=0.026) and FIG. 28E represents graph for tPA (white bars);

FIGS. 29A to 29E show the effect of Pumactant on gene transcription on surface derived cells (n=3). FIGS. 29A-29E represent the absolute gene regulation. FIG. 29A shows the surface-derived cells response to Pumactant for collagen type I (Grey bars); there was no statistical significance. FIG. 29B represents the graph for Collagen type III (stripe bars). FIG. 29C represents the graph for fibronectin gene expression (black bars). FIG. 29D represents the graph for PAI-1 (spots) and FIG. 29E represents the graph for tPA (white bars) where gene transcription showed a down regulation but no statistical change over the time course;

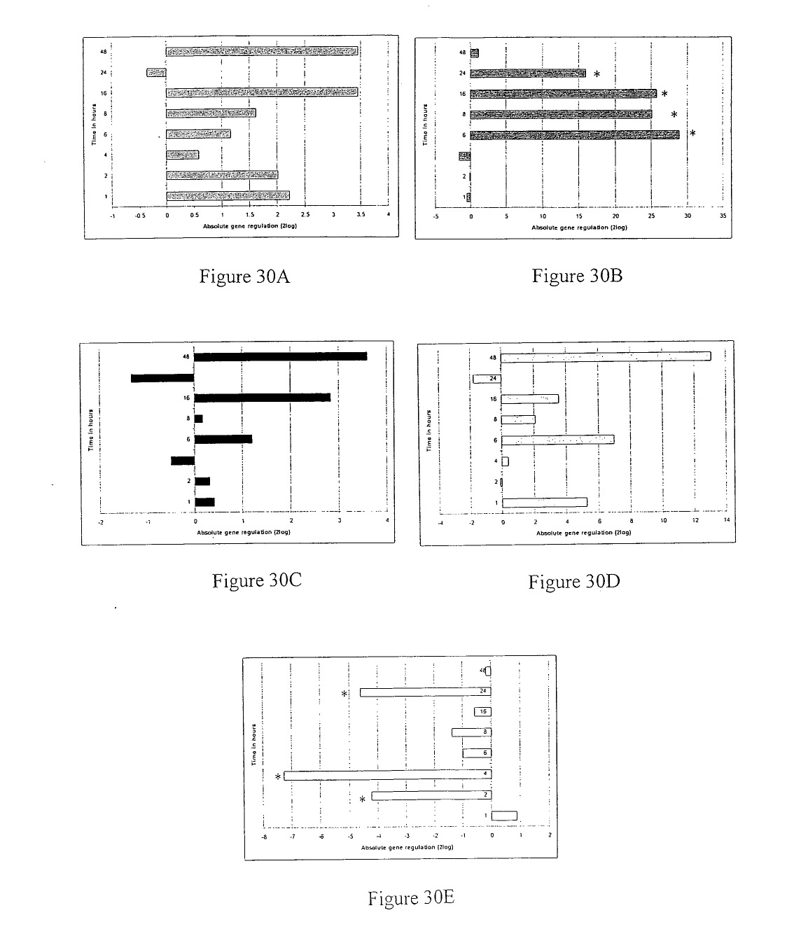

FIGS. 30A to 30E show TGF-β1 effect on gene transcription on sheath derived cells (n=3). FIG. 30A shows the sheath-derived cells response to TGF-β1 for collagen type I (Grey bars). FIG. 30B represents the graph for Collagen type III (stripe bars) which showed a statistically significant up regulation at 6 hrs (*p=0.03), 8 hrs (*p=0.037), 16 hrs (*p=0.012) and 24 hrs (*p=0.03), FIG. 30C represents the graph for fibronectin gene expression (black bars). FIG. 30D shows the graph for PAI-1 (spots) and FIG. 30E shows graph for tPA (white bars) which showed a statistically significant down regulation at 2 hrs (*p=0.013), 4 hrs (*p=0.001) and 24 hrs (*p=0.001);

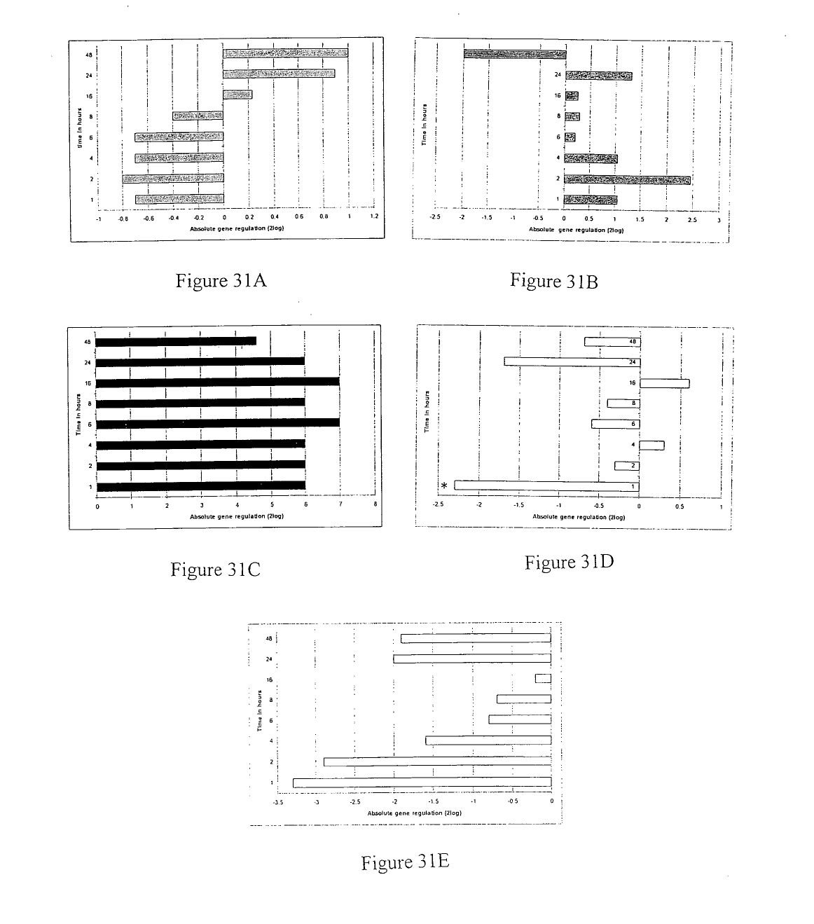

FIGS. 31A to 31E show the effect of Pumactant on gene transcription on sheath derived cells (n=3). FIGS. 31A to 31E represent the absolute gene regulation. FIG. 31A shows the sheath-derived cells response to Pumactant for collagen type I (Grey bars); there was no statistical significance. FIG. 31B represents the graph for Collagen type III (stripe bars). FIG. 31C represents the graph for fibronectin gene expression (black bars), which showed up regulation but there was no significant change over the time course. FIG. 31D the graph for PAI-1 (spots) which showed a statistically significant down regulation at 1 hour (*p=0.001) and FIG. 31E for tPA (white bars) gene transcription showed a down regulation but no statistical change over the time course;

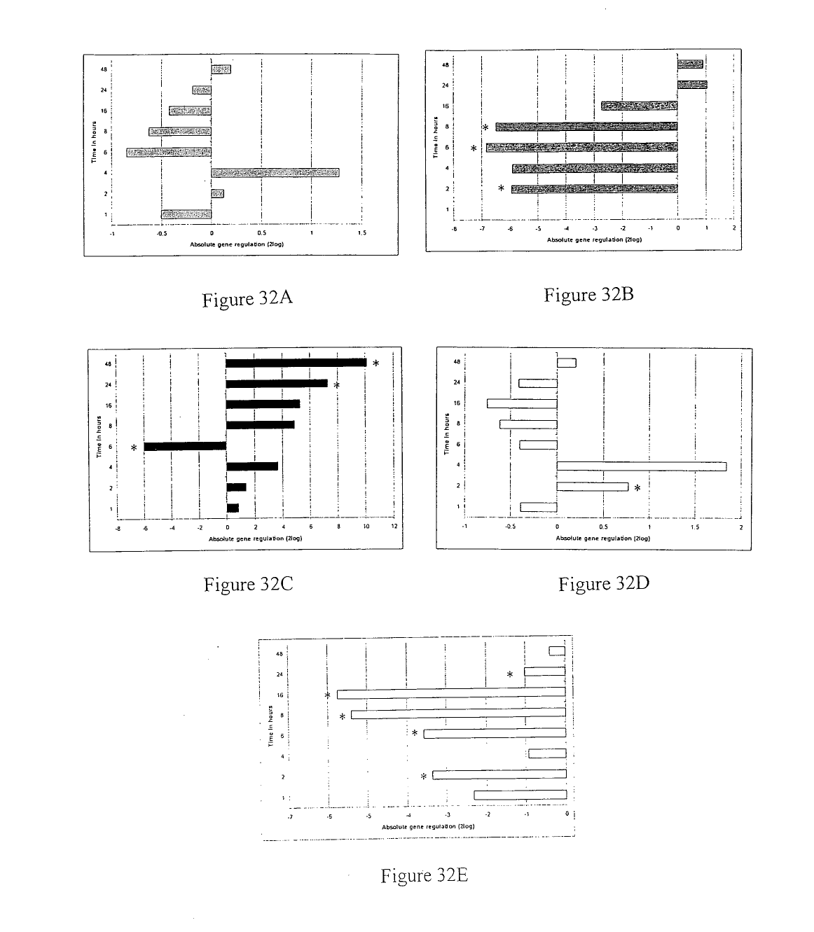

FIG. 32A to 32E show TGF-β1 effect on gene transcription on core derived cells (n=3). FIGS. 32A to 32E represent the absolute gene regulation. FIG. 32A shows the core-derived cells response to TGF-β1 for collagen type I (Grey bars). FIG. 32B represents the graph for Collagen type III (stripe bars) which showed a statistically significant down regulation at 2, 6 and 8 hours (*p=<0.02). FIG. 32C represents the graph for fibronectin gene expression (black bars), which showed a significant down regulation of fibronectin gene transcription at 6 hours (*p=0.024) and a statistically significant up-regulation at 24 hrs (*p=0.03) and 48 hrs (*p=0.04). FIG. 32D shows the graph for PAI-1 (spots) which showed a statistically significant increase at 2 hrs (*p=0.001). FIG. 32E shows the graph for tPA (white bars) which showed a statistically significant down regulation at 2 h, 6 h, 8 h, 16 h and 24 h;

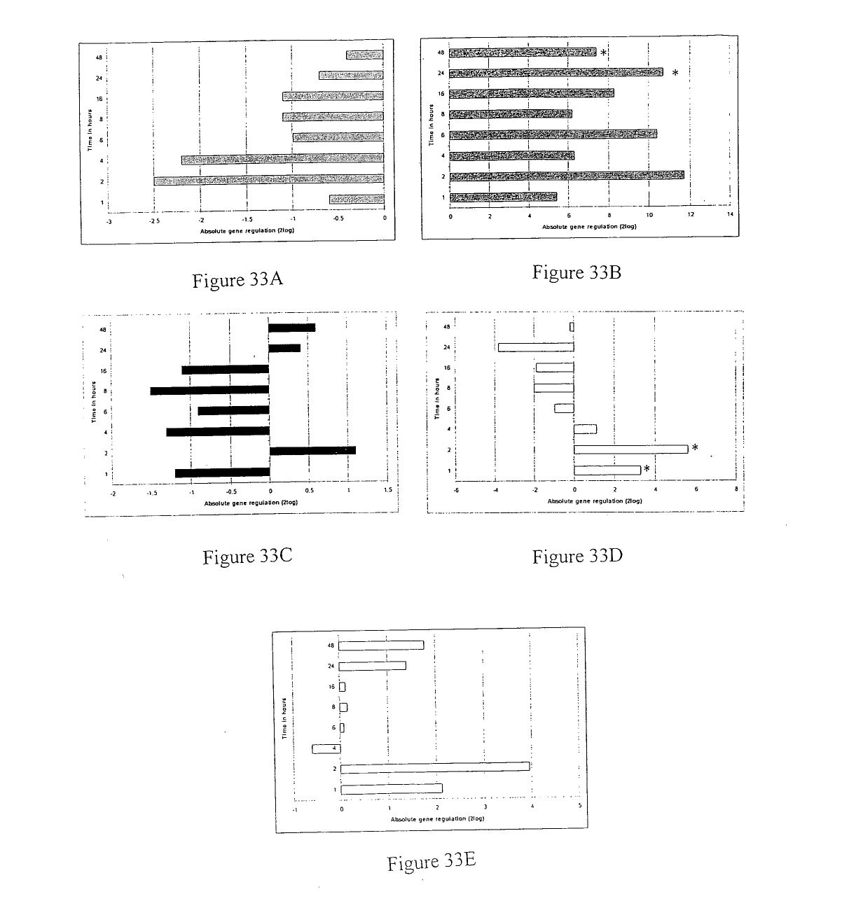

FIGS. 33A to 33E shows the effect of Pumactant (1 mg/ml) on gene transcription on core derived cells (n=3). FIGS. 33A to 33E represent the absolute gene regulation. FIG. 33A shows the core-derived cells response to Pumactant for collagen type I (Grey bars) which showed a down regulation but this was not statistically significant. FIG. 33B represents the graph for Collagen type III (stripe bars) that showed a statistically significant up regulation at 24 hrs and 48 hours (*p=0.025). FIG. 33C represents the graph for fibronectin gene expression (black bars), which showed no significant change over the time course. FIG. 33D shows the graph for PAI-1 (spots) which showed a statistically significant up regulation at 1 and 2 hrs (*p=0.001) and FIG. 33E shows the graph for tPA (white bars) where gene transcription showed no statistical change over the time course;

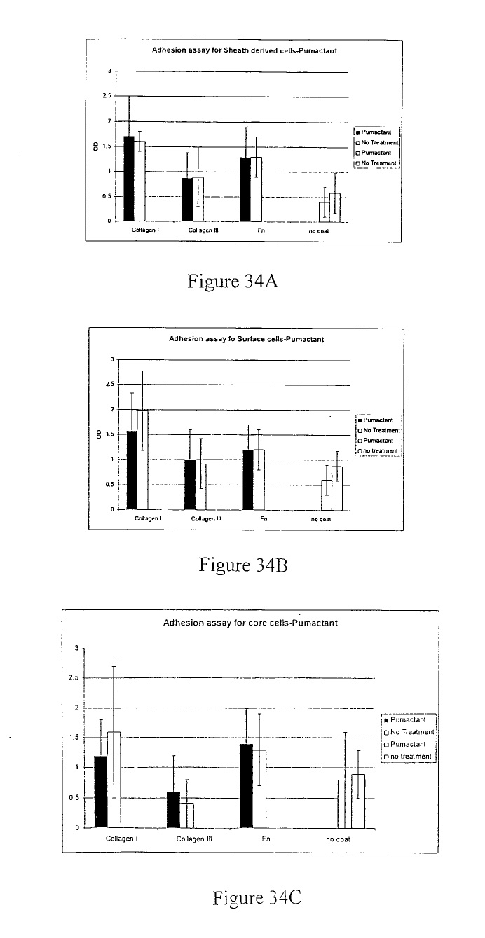

FIGS. 34A, 34B and 34C represent the mean for each cell type (n=3) error bars=SD. Adhesion assay following treatment with pumactant (1 mg/ml). FIG. 34A shows a graph of the results of an assay with sheath derived cells. FIG. 34B shows a graph of the results of an assay with surface cells. FIG. 34C shows a graph of the results of an assay with core cells. No cell type showed any statistical significant difference between the cells cultured with pumactant and those that did not;

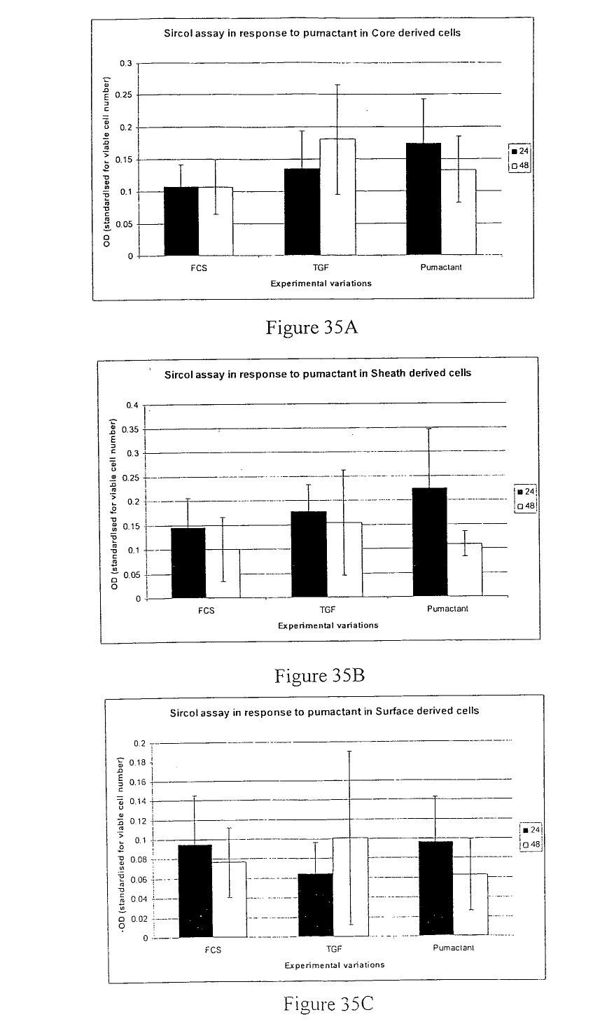

FIGS. 35A, 35B and 34B represent graphs represent the mean of each cell type (n=3) Error bars=SD. Sircol assay following treatment with pumactant (1 mg/ml). FIG. 35A shows a graph of the results of an assay with core derived cells. FIG. 35B shows a graph of the results of an assay with sheath derived cells. FIG. 34C shows a graph of the results of an assay with surface derived cells. No cell type showed any statistical significant change in soluble collagen production;

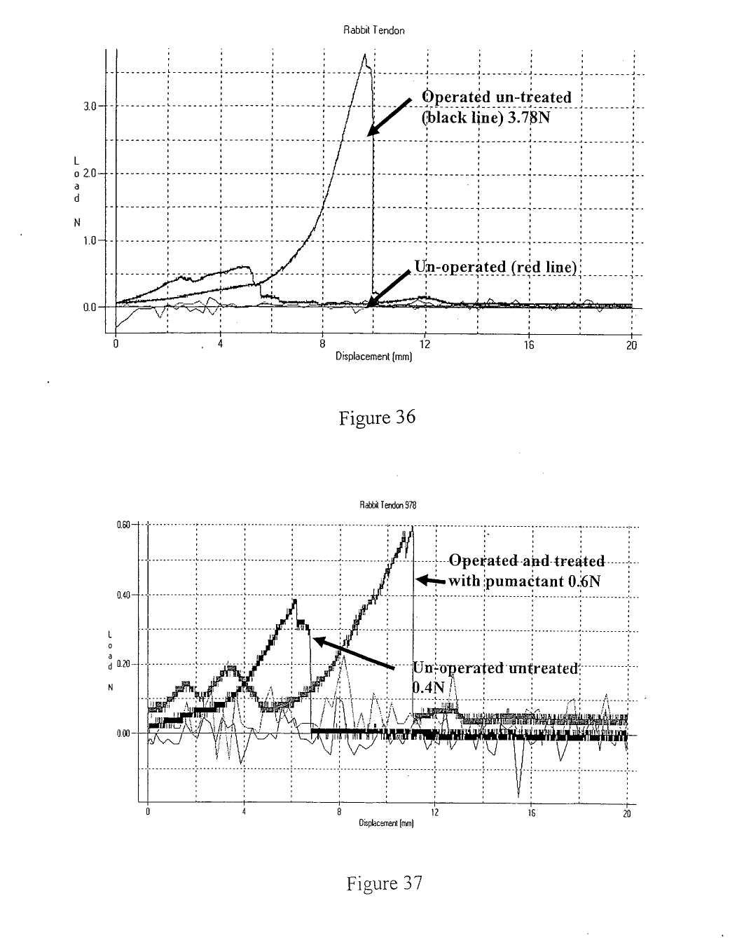

FIG. 36 shows a graph which is a typical example of the raw data from the mechanical tensile testing machine (Mecmesin) using the Emperor™ Software. It shows the load (force in Newtons) required to displace a rabbit tendon from the tendon sheath. The peak force (N) measured is the point when the adhesion fails. This graph compares the two control groups: operated untreated and un-operated untreated;

FIG. 37 shows a graph which is a typical example of the raw data from the mechanical tensile testing machine (Mecmesin) using the Emperor™ Software. It shows the load (force in Newtons) required to displace a rabbit tendon from the tendon sheath. The peak force (N) measured is the point when the adhesion fails. This graph compares the operated Pumactant treated group with the un-operated untreated group;

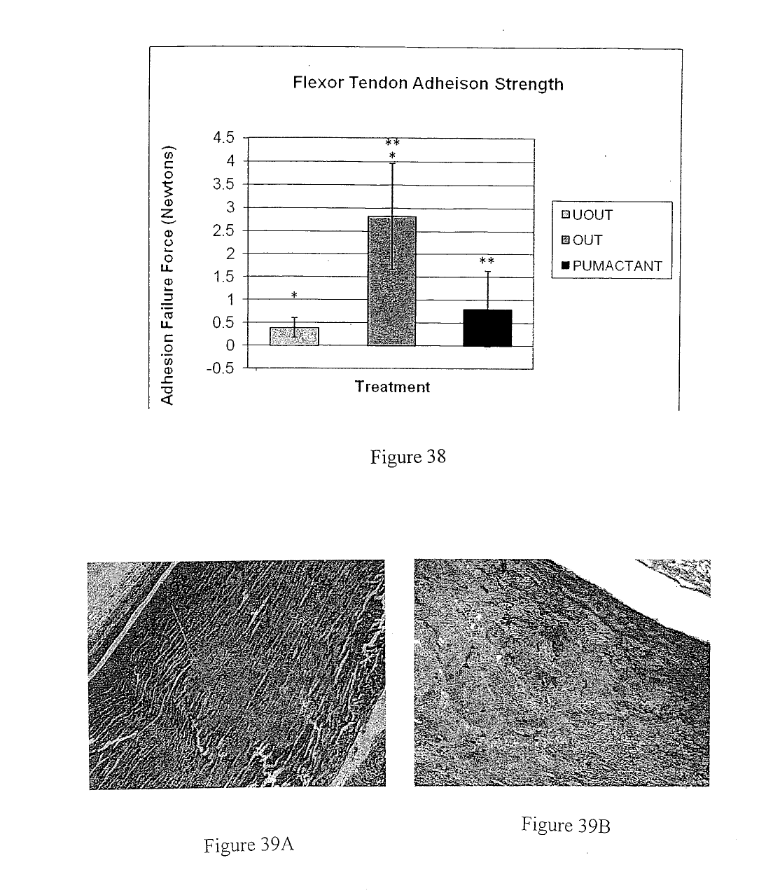

FIG. 38 shows a graph which represents the average pull out force for the experimental groups: un-operated (UOUT; n=16), operated un-treated (OUT; n=8), operated but treated with pumactant (n=3). Error bars=SEM. There was a statistically significant difference between the UOUT and OUT groups (*p<0.0001) and between the Pumactant and OUT groups (**p=0.0012);

FIGS. 39A and 39B show light microscopy photographs at ×40 magnification. FIG. 39A shows a photograph of unoperated untreated (UOUT) tendon, showing bundles of collagen fibrils stained blue (Masson's Trichrome Stain). FIG. 39B shows a photograph of injured tendon, showing blue (Masson's Trichrome Stain) collagen fibrils but also new matrix being laid down in the tendon (stained red);

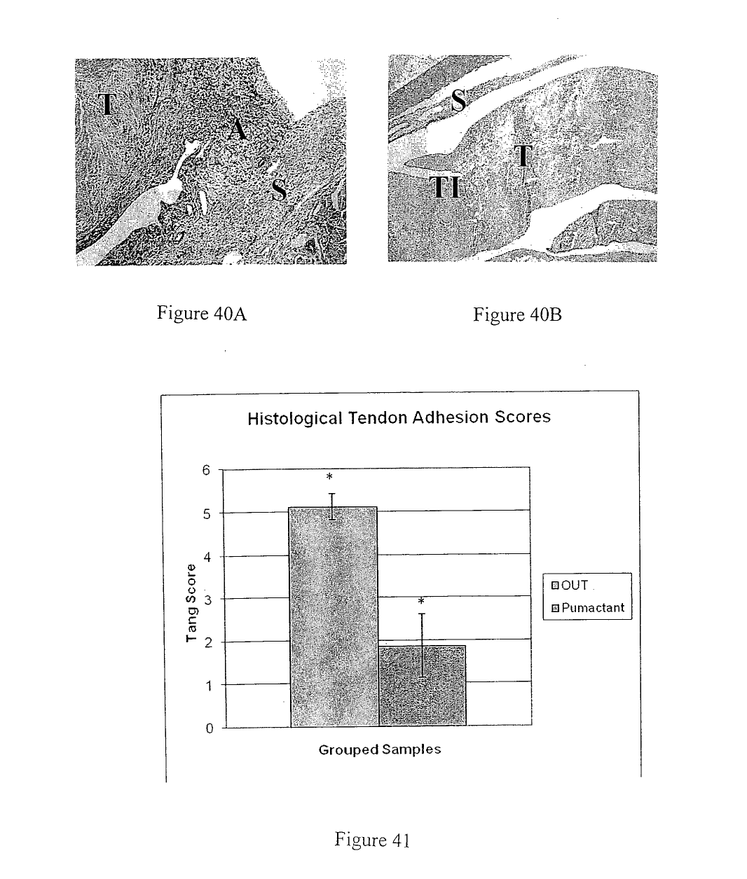

FIGS. 40A and 40B show light microscopy photographs at ×40 magnification. FIG. 40A shows a photograph showing an injured tendon without treatment with an obvious ‘adhesion’ bridge (A) between the tendon and the sheath. Legend: T=Tendon, A=Adhesion, S=Sheath. FIG. 40B shows a photograph showing an injured tendon treated with Pumactant with no evidence of adhesion formation. Legend: TI=Site of Tendon Injury, T=Tendon, S=Sheath; and

FIG. 41 shows a graph representing the average histological score based on Tang et al (1996) and error bars=SEM. There was a statistically significant difference (*p=0.0012) between the two groups.

The invention is illustrated by the following Examples which are not intended to limit the scope of the claims.

### EXAMPLE 1

In the following Example, the preparation of a phospholipid according to the invention is described. 100 vials each containing 300 mg of phospholipid were prepared.

An aqueous dispersion containing 0.75% w/v phospholipid was prepared by dissolving 30 g of phospholipid prepared as described in Example 1 in a vessel containing 0.4 litres of water at about 50° C.-60° C. by stirring for about 30 minutes.

The aqueous dispersion was homogenised in three cycles by means of a high pressure homogeniser operating at 960 bar.

The translucent homogenised aqueous dispersion was sterilised by filtration through a 0.2 μm membrane of cellulose acetate under pressure. A press filtration unit having a 2 litre volume was used under nitrogen gas. The filtration unit was filled with the homogenised aqueous dispersion at a temperature of 60° C. in order that the final temperature of the filtered homogenised aqueous dispersion was from 40° C. to 50° C. This temperature was selected because it is above the phase transition temperature of phospholipid prepared as described in Example 1 which makes filtration easier. After having closed the filtration unit, a pressure of 2-3 bar nitrogen was applied. The filtrate was collected in sterile bottles.

The sterilised homogenised aqueous dispersion was dispensed into 10 ml vials at a volume of 4 ml per vial such that each vial contained 300 mg of phospholipid.

The vials were then subjected to the following freeze drying cycle:
The vials were loaded into a freeze dryer having a shelf temperature of 8° C.;
The vials were frozen;
The shelf temperature was set to −40° C. and the vials were ramped cooled to this temperature at −1° C. per minute over 48 minutes;
The vials were held at −40° C. for 3 hours;
A vacuum was applied;
The vials were held at −40° C. for 45 minutes as the vacuum was applied to at least 0.1 mBar;
The shelf was warmed to 5° C. at a warming rate of 1° C. per minute over 45 minutes;
The vials were held at 5° C. for 20 hours for the main drying period;
The shelf was warmed to 16° C. at 1° C. per minute over 11 minutes;
The vials were held at 16° C. for 20 hours for the final drying period;
The shelf was cooled to 5° C. at 1° C. per minute over 11 minutes; and
The samples were held at 5° C. until stoppered under full vacuum before being removed from the freeze dryer.

### EXAMPLE 2

To prepare a phospholipid suitable for pulmonary administration, Example 1 is repeated without the sterilisation filtration step. Thus, after the aqueous dispersion is homogenised in three cycles by means of a high pressure homogeniser operating at 960 bar, the homogenised aqueous dispersion is dispensed into 10 ml vials at a volume of 4 ml per vial such that each vial contains 300 mg of phospholipid. The vials are then subjected to the freeze drying cycle set out in Example 1. The samples are then subjected to a micronisation step wherein they are micronized in a high pressure air jet mill to yield a phospholipid suitable for pulmonary administration, having a mass median aerodynamic diameter of less than 10 μm

As an alternative to the preparation method described in this Example, the method of Example 1 may be used to prepare a phospholipid suitable for pulmonary administration by repeating the method of Example but including a micronisation step as set out above.

### EXAMPLE 3

In the following Example, the effect of FCS, phospholipid prepared according to Example 1 (Vibrated and Non-vibrated), prior art phospholipid prepared by a method using an organic non-polar solvent to disperse the phospholipid prior to filtration (ALEC® dry powder (Britannia Pharmaceuticals Ltd)), lysophosphatidic acid (LPA), DPPC, DPPC/LPA, POPC, Curosurf and Intralipid on the rate of re-epithelialisation following scratch wounding (cell proliferation, migration) in human peritoneal mesothelial cells (HPMC), human keratinocytes and human lung epithelial cells was investigated.

The impact of phospholipids on re-epithelialisation (human peritoneal mesothelial cells and keratinocytes and lung epithelial cells) was assessed by time lapse photomicroscopy (as previously reported, Yung et al. 1994, 1998 and 2000, Morgan et al, 2003).

Mesothelial cells (HPMC) obtained from the omentum of healthy human donors by sequential trypsin digestion (Morgan et al. 2003) and Human Epidermal Keratinocytes (NHEK-A, TCS Cell Works, Buckingham, UK) were seeded onto 24-well culture plates. Following (48 hours) growth arrest, in serum-free medium, the monolayer was injured by mechanical linear scraping with a sterile pipette tip to leave a reproducible area devoid of cells. The well was washed with serum free medium to remove detached cells.

A phospholipid according to the invention reconstituted in M199 (at 37° C.), was directly applied to the HPMC and NHEK-A monolayers in serum free medium (1 mL). Controls used were serum free medium alone, or M199 medium supplemented with 2% (v/v) FCS (as a positive control). The denuded area in each well was identified microscopically and the co-ordinates recorded for subsequent data capture. Re-mesothelialisation was continuously monitored using an Axiovert 100M inverted microscope fitted with a computer-controlled XY automated scanning stage and incubator. The humidified incubator was maintained at 37° C. and 5% CO, with a heated insert and vectorial airflow (Carl Zeiss, Oberkochen, Germany). Images were captured from the same position in each well of the 24-well plates, using the 2.5× objective, at 60-minute intervals on an Orca C5985 digital video camera (Hamamatsu Photonics, Hamamatsu City, Japan). Images were analyzed using Openlab version 3.0.8 on a Macintosh G4 computer (Improvision, Ltd., Coventry, UK). The rate of re-mesothelialization was calculated by measuring the reduction of the denuded area (in pixels) at 60-minute intervals until wound closure was seen.

Previous data generated using prior art phospholipid (ALEC® dry powder) applied using the V-PAG device demonstrated a significant increase in the rate of HPMC wound closure compared to the endogenous (control process) but at a slower rate than that induced by 2% v/v FCS (FIG. 1). This data is disclosed in the international patent application publication no. WO 2006/056800.

Treatment of HPMC with a phospholipid prepared as described in Example 1 and resuspended in M199 (Gibco Product ref. 31150-022) at a maximum dissolved dose (100% dosage of 4 mg/ml), half of the maximum dissolved dose (50% dosage of 2 mg/ml) and one quarter of the maximum dissolved dose (25% dosage of 1 mg/ml). For the 100% and 50% dosages, a significant increase in wound closure in both cell types resulted (FIG. 2). In these cases, the rate of wound closure was greater than that induced by 2% v/v FCS.

Treatment of NHEK-A with the 100%, 50% and 25% dosages gave a similar pattern of results with wound closure at a rate greater than both the endogenous control and the 2% v/v FCS positive control for the 100% and 50% dosages (FIG. 3).

FIG. 4 shows the accelerated time course of wound closure in HPMC treated with 100% and 50% dosages in comparison to the control and 2% v/v FCS. There is visible wound closure when the wound area is about 50000 pixels. FIG. 4 shows that wound closure was complete after 7.5 hours for the 50% dosage and after 9 hours for the 100% dosage. After 10 hours, there was wound closure for 2% v/v FCS but not for the control.

Treatment of HPMC cells with a phospholipid prepared as described in Example 1 and resuspended in M199 (Gibco Product ref. 31150-022) at 25% of the maximum dissolved dose (1 mg/ml dosage), 12.5% of the maximum dissolved dose (0.5 mg/ml dosage), 6.25% of the maximum dissolved dose (0.25 mg/ml dosage), 3.125% of the maximum dissolved dose (0.125 mg/ml dosage) FIG. 5 shows the rate of change in the proliferation of HPMC cells treated with 3.125%, 6.25%, 12.5% and 25% dosages of a phospholipid prepared according to Example 1 in comparison to 2.5% v/v FCS in M199 medium.

The phospholipid prepared according to Example 1 reconstituted in liquid form surprisingly shows improved results compared to the known formulation. The phospholipid according to the invention accelerates the re-epithelialisation process in both mesothelial cells and keratinocytes. Based on the number of independent experiments performed with different donor cells (n=3 in each case), it is clear that application of the phospholipid according to the invention has an advantage over the known ALEC® dry powder formulation in promoting the wound healing process.

### EXAMPLE 4

The effect of a phospholipid prepared as described in Example 1 (and/or its constituent compounds) to induce human peritoneal mesothelial cell proliferation was assessed using an Alamar Blue™ assay and an MTT assay.

The Alamar Blue™ assay is designed to measure the proliferation of various cell types by measuring the ability of the cells to metabolise and reduce a REDOX indicator (Alamar Blue™). Reduction of the REDOX indicator results in a change in its fluorescent activity. Therefore the fluorescent activity measured is proportional to cell proliferation.

The MTT Assay measures the proliferations of various cells and is based on the mitochondrial conversion of tetrazolium salt 3-[4,5-dimethyl-thiazol-2-yl]-2,5-diphenyltetrazium bromide (MTT), into an insoluble formazan salt. The resultant colour change is directly related to the number of viable cells.

As described in Morgan L W et al (“Glucose degradation products (GDP) retard re-mesothelialisation independently of D-glucose concentration.” Kidney Int 64:1854-1866, 2003), primary HPMC are isolated from omental tissue biopsies donated by consenting patients undergoing elective abdominal surgery. Cells were grown to approximately 60% confluence in 96 well plates (Falcon) and used at passage 2. All cells were growth arrested in serum free M199 for 24 hours prior to use.

For the MTT assay, the following materials were used:

3-[4,5-dimethyl-thiazol-2-yl]-2,5-diphenyltetrazium bromide (Sigma)

Sodium lauryl sulphate (SDS) (Sigma)

PBS (Gibco)

N,N-Dimethylformamide

Sterile water

Acetic acid (glacial)

HCL

The supplemented M199 medium was prepared as follows. Earle's salt medium M199 was supplemented with 100μ/ml penicillin, 100 μg/ml streptomycin, 2 mM glutamine, 5 μg/ml insulin, 5 μg/ml transferrin and 0.4 μg/ml hydrocortisone and 2 mmol/L N-2-hydroxyethylpiperazine-N′-2ethanesulfonic acid.

The phospholipid prepared as described in Example 1 was prepared as follows. Using a Pumactant Delivery Device (Britannia Pharmaceuticals Ltd, Redhill UK) set at a pressure of 22 Kpa and a delivery time of 2.5 seconds, approximately 5 mg (single dose) of phospholipid prepared as described in Example 1 was added to 1 ml (per sample) of supplemented M199 medium (37° C.) in a 25 ml universal. Following brief agitation (by vortexing) the sample was then incubated for 1 hour at 37° C. Sample doses of 1/4× and 1/2× dilutions were prepared using appropriate dilutions of the solution described above.

An MTT solution (5 mg/ml) was prepared as follows. 100 mg of MTT was dissolved in 20 ml of sterile PBS. The solution was then vortexed and stored in a light resistant bottle.

Lysis buffer solution for the MTT assay was prepared by mixing 20 g SDS with 50 ml N,N-Dimethylformamide and 50 ml sterile water. The pH of the solution was adjusted to 4.7 (measured using a pH meter) by the addition of the appropriate amount of an acid mix formed from 80 ml of acetic acid, 2.5 ml of 1N HCL and 17.5 ml of sterile water.

### Methods

A volume of 200 μl per well was used per stimulation. The nature of each stimulation is shown in FIGS. 6A-6F, 7A-7F, 8A-8F, 9A-9F and 10A-10F, as discussed below. A positive control of supplemented M199 with a defined concentration of FCS was used as well as a negative control of supplemented M199. Each stimulation was performed in triplicate at each time point.

The growth medium was aspirated from each well and washed once using 200 μl supplemented medium. To an allocated well was added 200 μl of the each stimulation to separate wells in triplicate.

The plates were incubated in tissue culture incubator. At each time point, the HPMC assay was conducted for proliferation using Alamar Blue™ and/or MTT. The MTT assay can be conducted following Alamar Blue™ treatment due to the non-toxic nature of the Alamar Blue™ reagent.

### Alamar Blue™ Assay

An appropriate amount of Alamar Blue™ reagent was added to supplemented M199 to produce a 10% (v/v) Alamar Blue™ solution.

The growth medium was aspirated from each well and then the well was washed once using 200 μl per well of supplemented M199. To each well was added 200 μl per well of the previously prepared 10% Alamar Blue™ solution. The plate was incubated at 37° C. under 5% CO₂ for 1 hour. A 100 μl sample was transferred from each well to the corresponding well of a Microfluor 1 black flat bottom microtitre plate. The fluorescent activity of Alamar Blue™ at 540 nm (wavelength) was measured using a Dynex Revalation 96-multiwell plate reader. The results were normalised to the un-stimulated negative control value (C) for each time point using FCS standard curve such as that shown in FIG. 14 and expressed in “Arbitrary Units”.

### MTT Assay

The growth medium was aspirated from each well and then each well was washed once using 200 μl per well of supplemented M199 (where this assay was used following the use of the Alamar Blue™ assay this step was replaced with a step of washing each well with 200 μl warm PBS). Using a multi-channel pipette, 100 μl of the previously made MTT solution was added to each well. The plate was incubated at 37° C. under 5% CO₂ for 4 hours (in the dark). After incubation, 1000 of lysis buffer was added to each well. The plate was then incubated at 37° C. under 5% CO₂ overnight. The absorbance at 600 nm (wavelength) was measured using the Dynex Revalation 96-multiwell plate reader. The results were interpreted by normalising the absorbance/fluorescence values to the un-stimulated control value (C) for each time point and are expressed as “Relative Fluorescence/Absorbance” or “Arbitrary Units”. The correlation between the Alamar Blue™ data and the MTT data is shown in FIG. 15.

### Results

Results from the HPMC assays were obtained after 48 hours and after 72 hours.

As a positive control FCS in supplemented M199 was used to stimulate HPMC and the data obtained at 48 hours and 72 hours is shown in the bar charts of FIGS. 6A and 6D, respectively. The data for FIG. 6A were from 12 wells whereas the data for FIG. 6D were from 11 wells. In these bar charts, the first bar from the left is for the negative control (C). FCS was used at concentrations (v/v %) of 0.156, 0.313, 0.625, 1.25 and 2.5 and the results obtained for each concentration are shown in the respective adjacent bars of the bar charts. The results in FIGS. 6A and 6D show a significant dose dependent proliferation response to FCS compared to the negative control.

Vibrated phospholipid prepared as described in Example 1 and dispersed in supplemented M199 was used to stimulate HPMC and the data obtained at 48 hours and 72 hours is shown in the bar charts of FIGS. 6B and 6E, respectively. The data for FIGS. 6B and 6E were each from 6 wells. In these bar charts, the first bar from the left is for the negative control (C). Vibrated phospholipid prepared as described in Example 1 was used at concentrations (mg/ml) of 0.125, 0.25, 0.5, 1, 2 and 4 and the results obtained for each concentration are shown in the respective adjacent bars of the bar charts. The results in FIGS. 6B and 6E show that compared to the negative control, vibrated phospholipid prepared as described in Example 1 significantly stimulated HPMC proliferation, particularly at a concentration of from 0.125 to 2 mg/ml at 48 hours and at a concentration of from 0.125 to 1 mg/ml at 72 hours. The results show that the rate of proliferation accelerates over time.

Non-vibrated phospholipid prepared as described in Example 1 and dispersed in supplemented M199 was used to stimulate HPMC and the data obtained at 48 hours and 72 hours is shown in the bar charts of FIGS. 6C and 6F, respectively. The data for FIGS. 6C and 6F were each from 4 wells. In these bar charts, the first bar from the left is for the negative control (C). Non-vibrated phospholipid prepared as described in Example 1 was used at concentrations (mg/ml) of 0.125, 0.25, 0.5, 1, 2 and 4 and the results obtained for each concentration are shown in the respective adjacent bars of the bar charts. The results in FIGS. 6C and 6F show that compared to the negative control, non-vibrated phospholipid prepared as described in Example 1 significantly stimulated HPMC proliferation, particularly at a concentration of from 0.125 to 1 mg/ml at 48 hours and at a concentration of from 0.125 to 0.5 mg/ml at 72 hours.

Lysophosphatidic acid (LPA) in supplemented M199 was used to stimulate HPMC and the data obtained at 48 hours and 72 hours is shown in the bar charts of FIGS. 7A and 7D, respectively. The data for FIG. 7A were from 3 wells whereas the data for FIG. 7D were from 4 wells. In these bar charts, the first bar from the left is for the negative control (C). LPA was used at concentrations (μg/ml) of 2.5, 5, 10, 20, 40 and 80 and the results obtained for each concentration are shown in the respective adjacent bars of the bar charts. The results show no significant stimulation of HPMC proliferation compared to the negative control.

Dipalmitoyl phosphatidyl choline (DPPC) in supplemented M199 was used to stimulate HPMC and the data obtained at 48 hours and 72 hours is shown in the bar charts of FIGS. 7B and 7E, respectively. The data for FIG. 7B were from 3 wells whereas the data for FIG. 7E were from 4 wells. In these bar charts, the first bar from the left is for the negative control (C). DPPC was used at concentrations (mg/ml) of 0.0875, 0.175, 0.35, 0.7, 1.4 and 2.8 and the results obtained for each concentration are shown in the respective adjacent bars of the bar charts. The results show no significant stimulation of HPMC proliferation compared to the negative control.

A mixture of DPPC and LPA in supplemented M199 was used to stimulate HPMC and the data obtained at 48 hours and 72 hours is shown in the bar charts of FIGS. 7C and 7F, respectively. The data for FIGS. 7C and 7F were each from 3 wells. In these bar charts, the first bar from the left is for the negative control (C). A DPPC/LPA mixture was used at respective concentrations ((mg/ml)/(μg/ml)) of 0.0875/2.5, 0.175/5, 0.35/10, 0.7/20, 1.4/40 and 2.8/80 and the results obtained for each concentration are shown in the respective adjacent bars of the bar charts. The results show no significant stimulation of HPMC proliferation compared to the negative control.

Palmitoyl oleoyl phosphatidyl choline (POPC) in supplemented M199 was used to stimulate HPMC and the data obtained at 48 hours and 72 hours is shown in the bar charts of FIGS. 8A and 8D, respectively.

The data for FIGS. 8A and 8D were each from 3 wells. In these bar charts, the first bar from the left is for the negative control (C). POPC was used at concentrations (mg/ml) of 0.0875, 0.175, 0.35, 0.7, 1.4 and 2.8 and the results obtained for each concentration are shown in the respective adjacent bars of the bar charts. The results show no significant stimulation of HPMC proliferation compared to the negative control.

Curosurf™ (Chiesi Farmaceutici)) in supplemented M199 was used to stimulate HPMC and the data obtained at 48 hours and 72 hours is shown in the bar charts of FIGS. 8B and 8E, respectively. The data for FIGS. 8B and 8E were each from 3 wells. In these bar charts, the first bar from the left is for the negative control (C). Curosurf™ was used at concentrations (μg/ml) of 31.25, 62.5, 125, 250, 500 and 1000 and the results obtained for each concentration are shown in the respective adjacent bars of the bar charts. The results show a significant decrease in HPMC proliferation compared to the negative control at concentrations of 62.5 to 1000 μg/ml at 48 hours and a significant decrease in HPMC proliferation compared to the negative control at all tested concentrations at 72 hours.

Egg-PG (EPG) in supplemented M199 was used to stimulate HPMC and the data obtained at 48 hours and 72 hours is shown in the bar charts of FIGS. 8C and 8F, respectively. The data for FIGS. 8C and 8F were each from 3 wells. In these bar charts, the first bar from the left is for the negative control (C). EPG was used at concentrations (mg/ml) of 0.0875, 0.175, 0.35, 0.7, 1.4 and 2.8 and the results obtained for each concentration are shown in the respective adjacent bars of the bar charts. The results show a significant decrease in HPMC proliferation compared to the negative control at concentrations of 0.35 to 2.8 mg/ml at 48 hours and a significant decrease in HPMC proliferation compared to the negative control at all tested concentrations at 72 hours.

Intralipid™-10% (Fresenius Kabi, Germany) in supplemented M199 was used to stimulate HPMC and the data obtained at 48 hours and at 72 hours is shown in the bar charts of FIGS. 9A and 9D, respectively. Intralipid™-10% is a 10% v/v suspension of lipids in water. In particular, Intralipid™-10% contains 50 g of soybean oil, 6 g of lecithin, 11.25 g of glycerine and 430.5 g of water. The data for FIG. 9A were from 3 wells whereas the data for FIG. 9D were 4 wells. In these bar charts, the first bar from the left is for the negative control (C). Intralipid™-10% was used at v/v % concentrations of 3.125%, 6.25%, 12.5%, 25%, 50% and 100% in serum-free tissue culture medium (M199) and the results obtained for each concentration are shown in the respective adjacent bars of the bar charts. The results show a significant decrease in HPMC proliferation compared to the negative control at all tested concentrations at both 48 hours and 72 hours.

Intralipid™-20% (Fresenius Kabi, Germany) in supplemented M199 was used to stimulate HPMC and the data obtained at 48 and 72 hours is shown in the bar charts of FIGS. 9B and 9E, respectively. Intralipid™-20% is a 20% v/v suspension of lipids in water having similar ingredients to Intralipid™-10%. The data for FIG. 9B were from 3 wells whereas the data for FIG. 9E were 4 wells. In these bar charts, the first bar from the left is for the negative control (C). Intralipid™-20% was used at v/v % concentrations of 3.125%, 6.25%, 12.5%, 25%, 50% and 100% in serum-free tissue culture medium (M199) and the results obtained for each concentration are shown in the respective adjacent bars of the bar charts. The results show a significant decrease in HPMC proliferation compared to the negative control at all tested concentrations at both 48 hours and 72 hours.

Intralipid™-30% (Fresenius Kabi, Germany) in supplemented M199 was used to stimulate HPMC and the data obtained at 48 hours and at 72 hours is shown in the bar charts of FIGS. 9C and 9F, respectively. Intralipid™-30% is a 30% v/v suspension of lipids in water having similar ingredients to Intralipid™-10%. The data for FIG. 9C were from 3 wells whereas the data for FIG. 9F were 4 wells. In these bar charts, the first bar from the left is for the negative control (C). Intralipid™-30% was used at v/v % concentrations of 3.125%, 6.25%, 12.5%, 25%, 50% and 100% in serum-free tissue culture medium (M199) and the results obtained for each concentration are shown in the respective adjacent bars of the bar charts. The results show a significant decrease in HPMC proliferation compared to the negative control at concentrations of from 3.12% to 25% and 100% at 48 hours and a significant decrease in HPMC proliferation compared to the negative control at concentrations of from 3.12% to 12.5% and 100% at 72 hours.

### EXAMPLE 5

The viability of HPMC after stimulation with FCS, phospholipid prepared according to Example 1 (Vibrated and Non-vibrated), lysophosphatidic acid (LPA), DPPC, DPPC/LPA, POPC, Curosurf, egg-PG (EPG) and the three Intralipid™ products was determined using an ATP assay. This assay measures the light emitted during the ATP dependant oxidation of luciferin in order to accurately quantify the ATP concentration.

As described in Example 3, primary HPMC are isolated from omental tissue biopsies donated by consenting patients undergoing elective abdominal surgery. Cells are grown to approximately 60% confluence in 96 well plates (Falcon) and used at passage 2. All cells are growth arrested in serum free M199 for 24 hours prior to use.

An ATP Bioluminescence Assay Kit CLS II (Roche Diagnostics, Cat No 1 699 695, 11857300) was used. It contains ATP Standard, Luciferase and distilled water. A 10 mM EDTA solution was prepared by dissolving 1.8612 g EDTA (Fischer Scientific, Code D/0650/60, Batch 0447430, Mr-372.24) in 500 ml H₂O (or 3.7224 g in 1 litre). A BAC-extract solution was prepared by dissolving 1 g Benzalkonium Chloride (Sigma-Aldrich, Cat:-23, 442-7, Lot S22104-334) in 1 litre of 10 mM EDTA. A 25 mM Hepes solution was prepared by dissolving 2.9788 g Hepes (Sigma, H3375-25G, Batch 094K5432, Mr-238.31) in 500 ml of 10 mM EDTA (or 5.9577 g Hepes in 1 litre).

Prior to the following procedure, HPMC used in Example 3 (Alamar Blue™ Proliferation study) were washed in PBS and lysed using 1 mg/ml BAC-extract and 25 mM Hepes. Samples were then frozen and stored at −20° C. until required.

Before conducting the ATP Assay, a standard curve needs to be prepared using the following steps:
1) Add 10 ml of distilled water to luciferase reagent in bioluminescence kit and leave to stand for 5 minutes (Do Not Shake). To mix, rotate the bottle carefully.
2) Make up an ATP standard solution having a base concentration of 1.65 μM ATP. Prepare a serial dilution of the 1.65 μM ATP (=1650 nM) using solution distilled water until a concentration range of 1650-0.0126 nM of ATP is obtained.
3) Decide on the concentration range for the standard curve e.g. 206.25 to 0.0126 nM and aliquot 20 μl of standard into the wells of a white 96 well plate in triplicate as on the template below. Use distilled water as the control.
4) Set the Fluostar Optima plate reader to measure luminescence. Set the Fluostar Optima plate reader pump to inject 20 μl of luciferase into each of the required wells.
5) Place the plate into the plate reader and measure the relative light units (RLU).
6) Prepare a standard curve such as that shown in FIG. 16 by plotting a graph of RLU against ATP concentration.

96 well plates from the HPMC proliferation study were defrosted. A 40 μl of sample from each well was transferred into the corresponding well of a white 96 well plate. Steps 4 to 6 of the ATP standard curve procedure were followed to measure the RLU of the samples. The ATP concentrations were calculated from the RLU of the samples using the standard curve. As an alternative to using an ATP standard curve, data can be normalised to the un-stimulated control and expressed as “ATP (% of control)”.

### Results

The results from the assays after 72 hours are shown in FIGS. 11A-11L. The data for each assay were from a single well.

As a positive control, FCS in supplemented M199 was used to stimulate HPMC and the data obtained is shown in the bar chart of FIG. 11A. In this bar chart, the first bar from the left is for the negative control (C). FCS was used at concentrations (v/v %) of 0.156, 0.312, 0.625, 1.25 and 2.5 and the results obtained for each concentration are shown in the respective adjacent bars of the bar chart. The results in FIG. 11A show a dose dependent proliferation response to FCS compared to the negative control.

Vibrated phospholipid prepared as described in Example 1 and dispersed in supplemented M199 was used to stimulate HPMC and the data obtained is shown in the bar chart of FIG. 11B. In this bar chart, the first bar from the left is for the negative control (C). Vibrated phospholipid prepared as described in Example 1 was used at concentrations (mg/ml) of 0.125, 0.25, 0.5, 1, 2 and 4 and the results obtained for each concentration are shown in the respective adjacent bars of the bar chart. The results in FIG. 11B show that compared to the negative control, vibrated phospholipid prepared as described in Example 1 increased HPMC proliferation at a concentration of from 0.125 to 0.25 mg/ml.

Non-vibrated phospholipid prepared as described in Example 1 and dispersed in supplemented M199 was used to stimulate HPMC and the data obtained is shown in the bar chart of FIG. 11C. In this bar chart, the first bar from the left is for the negative control (C). Non-vibrated phospholipid prepared as described in Example 1 was used at concentrations (mg/ml) of 0.125, 0.25, 0.5, 1, 2 and 4 and the results obtained for each concentration are shown in the respective adjacent bars of the bar chart. The results in FIG. 11C show that compared to the negative control, non-vibrated phospholipid prepared as described in Example 1 increased HPMC proliferation at a concentration of from 0.125 to 0.5 mg/ml. Non-vibrated phospholipid prepared as described in Example 1 had a greater effect on increasing HPMC proliferation than vibrated phospholipid prepared as described in Example 1.

Lysophosphatidic acid (LPA) in supplemented M199 was used to stimulate HPMC and the data obtained is shown in the bar chart of FIG. 11D. In this bar chart, the first bar from the left is for the negative control (C). LPA was used at concentrations (μg/ml) of 2.5, 5, 10, 20, 40 and 80 and the results obtained for each concentration are shown in the respective adjacent bars of the bar chart. The results show no increase of HPMC proliferation by LPA compared to the negative control.

Dipalmitoyl phosphatidyl choline (DPPC) in supplemented M199 was used to stimulate HPMC and the data obtained is shown in the bar chart of FIG. 11E. In this bar chart, the first bar from the left is for the negative control (C). DPPC was used at concentrations (mg/ml) of 0.0875, 0.175, 0.35, 0.7, 1.4 and 2.8 and the results obtained for each concentration are shown in the respective adjacent bars of the bar chart. The results show no increase of HPMC proliferation by DPPC compared to the negative control.

A mixture of DPPC and LPA in supplemented M199 was used to stimulate HPMC and the data obtained is shown in the bar chart of FIG. 11F. In this bar chart, the first bar from the left is for the negative control (C). A DPPC/LPA mixture was used at respective concentrations ((mg/ml)/(n/ml)) of 0.0875/2.5, 0.175/5, 0.35/10, 0.7/20, 1.4/40 and 2.8/80 and the results obtained for each mixture of concentrations are shown in the respective adjacent bars of the bar chart. The results show no increase of HPMC proliferation compared to the negative control by the DPPC/LPA mixture.

Palmitoyl oleoyl phosphatidyl choline (POPC) in supplemented M199 was used to stimulate HPMC and the data obtained is shown in the bar chart of FIG. 11G. In this bar chart, the first bar from the left is for the negative control (C). POPC was used at concentrations (mg/ml) of 0.0875, 0.175, 0.35, 0.7, 1.4 and 2.8 and the results obtained for each concentration are shown in the respective adjacent bars of the bar chart. The results show that POPC increased HPMC proliferation compared to the negative control at a concentration of 0.0875 mg/ml.

Curosurf™ (Chiesi Farmaceutici) in supplemented M199 was used to stimulate HPMC and the data obtained is shown in the bar chart of FIG. 11H. In this bar chart, the first bar from the left is for the negative control (C). Curosurf™ was used at concentrations (μg/ml) of 31.25, 62.5, 125, 250, 500 and 1000 and the results obtained for each concentration are shown in the respective adjacent bars of the bar chart. The results show a decrease in HPMC proliferation compared to the negative control by the Curosurf™ treatment.

Egg-PG (EPG) in supplemented M199 was used to stimulate HPMC and the data obtained is shown in the bar chart of FIG. 11I. In this bar chart, the first bar from the left is for the negative control (C). EPG was used at concentrations (mg/ml) of 0.0875, 0.175, 0.35, 0.7, 1.4 and 2.8 and the results obtained for each concentration are shown in the respective adjacent bars of the bar chart. The results show a decrease in HPMC proliferation compared to the negative control by EPG.

Intralipid™-10% (Fresenius Kabi, Germany) in supplemented M199 was used to stimulate HPMC and the data obtained is shown in the bar chart of FIG. 11J. In this bar chart, the first bar from the left is for the negative control (C). Intralipid™-10% was used at v/v % concentrations of 3.125%, 6.25%, 12.5%, 25%, 50% and 100% of a 10% v/v fat emulsion of Intralipid™-10% in serum-free tissue culture medium (M199) and the results obtained for each concentration are shown in the respective adjacent bars of the bar chart. The results show a decrease in HPMC proliferation compared to the negative control by Intralipid™-10%.

Intralipid™-20% (Fresenius Kabi, Germany) in supplemented M199 was used to stimulate HPMC and the data obtained is shown in the bar chart of FIG. 11K. In this bar chart, the first bar from the left is for the negative control (C). Intralipid™-20% was used at v/v % concentrations of 3.125%, 6.25%, 12.5%, 25%, 50% and 100% of a 20% v/v fat emulsion of Intralipid™-20% in serum-free tissue culture medium (M199) and the results obtained for each concentration are shown in the respective adjacent bars of the bar chart. The results show a decrease in HPMC proliferation compared to the negative control by Intralipid™-20%.

Intralipid™-30% (Fresenius Kabi, Germany) in supplemented M199 was used to stimulate HPMC and the data obtained is shown in the bar chart of FIG. 11L. In this bar chart, the first bar from the left is for the negative control (C). Intralipid™-30% was used at v/v % concentrations of 3.125%, 6.25%, 12.5%, 25%, 50% and 100% of a 30% v/v fat emulsion of Intralipid™-30% in serum-free tissue culture medium (M199) and the results obtained for each concentration are shown in the respective adjacent bars of the bar chart. The results show a decrease in HPMC proliferation compared to the negative control by Intralipid™-30%.

### EXAMPLE 6

The effect of phospholipid prepared as described in Example 1 or commercially available DPPC to induce human epidermal keratinocyte cell proliferation was assessed using the Alamar Blue™ assay and MTT assay.

The experiments were performed using a human epidermal keratinocyte cell line (TCS CellWorks). Cells were grown to approximately 60% confluence in 96 well plates (Falcon) and used at passage 2. All cells were growth arrested in serum free keratinocyte basal medium (TCS CellWorks) for 24 hours prior to use.

For the MTT assay, the following materials described in Example 3 were used.

Supplemented serum free keratinocyte basal medium was prepared by adding 1% FCS and the epidermal keratinocyte growth supplement pack (TCS CellWorks) to epidermal keratinocyte basal medium (TCS CellWorks). The epidermal keratinocyte growth supplement pack includes bovine insulin, hydrocortisone, bovine transferrin, human epidermal growth factor, bovine pituitary extract, 25 μg/ml gentamicin and 25 μg/ml amphotericin B.

The phospholipid was prepared as follows. Using the Pumactant Delivery Device (Pressure: 22 Kpa, Delivery time: 2.5 seconds) approximately 5 mg (single dose) of phospholipid prepared as described in Example 1 was added to 1 ml (per sample) of supplemented keratinocyte basal medium (37° C.) in a 25 ml universal. Following brief agitation (by vortexing) the sample was then incubated for 1 hour at 37° C. Sample doses of 1/4× and 1/2× dilutions were prepared using appropriate dilutions of the solution described above.

The MTT solution and the lysis buffer solution were prepared as described in Example 3.

### Methods

A volume of 200 μl per well was used per stimulation. The nature of each stimulation is shown in FIGS. 12A-12F, as discussed below. A positive control of supplemented keratinocyte basal medium with defined concentrations of FCS was used as well as a negative control of supplemented keratinocyte basal medium. Each stimulation was performed in triplicate at each time point.

The growth medium was aspirated from each well and washed once using 200 μl supplemented medium. To an allocated well was added 200 μl of each stimulation to separate wells in triplicate.

The plates were incubated in tissue culture incubator. At each time point, the NHEK-A assay was conducted for proliferation using Alamar Blue™ and/or MTT. The MTT assay can be conducted following Alamar Blue™ treatment due to the non-toxic nature of the Alamar Blue™ reagent.

### Alamar Blue™ Assay

An appropriate amount of Alamar Blue™ reagent was added to supplemented keratinocyte basal medium to produce a 10% (v/v) Alamar Blue™ solution.

The growth medium was aspirated from each well and then the well was washed once using 200 μl per well of supplemented keratinocyte basal medium. To each well was added 200 μl per well of the previously prepared 10% Alamar Blue™ solution. The plate was incubated at 37° C. under 5% CO, for 1 hour. A 100 μl sample was transferred from each well to the corresponding well of a Microfluor 1 black flat bottom microtitre plate. The fluorescent activity of Alamar Blue™ at 540 nm (wavelength) was measured using a Dynex Revalation 96-multiwell plate reader. The results were normalised to the un-stimulated negative control value (C) for each time point and expressed in “Arbitrary Units”.

### MTT Assay

The growth medium was aspirated from each well and then each well was washed once using 200 μl per well of supplemented keratinocyte basal medium (where this assay was used following the use of the Alamar Blue™ assay this step was replaced with a step of washing each well with 200 μl warm PBS). Using a multi-channel pipette, 100 μl of the previously made MTT solution was added to each well. The plate was incubated at 37° C. under 5% CO₂ for 4 hours (in the dark). After incubation, 100 μl of lysis buffer was added to each well. The plate was then incubated at 37° C. under 5% CO₂ overnight. The absorbance at 600 nm (wavelength) was measured using the Dynex Revalation 96-multiwell plate reader. The results were interpreted by normalising the absorbance/fluorescence values to the un-stimulated control value (C) for each time point and are expressed as “Relative Fluorescence/Absorbance” or “Arbitrary Units”.

### Results

As a positive control, FCS in supplemented keratinocyte basal medium was used to stimulate NEKa keratinocyte cells and the data obtained from the Alamar Blue™ assay after 48 hours and 72 hours are shown in the bar charts of FIGS. 12A and 12D, respectively. In these bar charts, the first bar from the left is for the negative control (C). FCS was used at concentrations (v/v %) of 0.062, 0.125, 0.25, 0.5 and 1.00 and the results obtained for each concentration are shown in the respective adjacent bars of the bar charts. The results show a dose dependent proliferation response to FCS compared to the negative control.

Vibrated phospholipid prepared as described in Example 1 and dispersed in supplemented keratinocyte basal medium was used to stimulate NEKa keratinocyte cells and the data obtained from the Alamar Blue™ assay after 48 hours and 72 hours are shown in the bar charts of FIGS. 12B and 12E, respectively. In these bar charts, the first bar from the left is for the negative control (C). Vibrated phospholipid prepared as described in Example 1 was used at concentrations (mg/ml) of 0.125, 0.25, 0.5, 1, 2 and 4 and the results obtained for each concentration are shown in the respective adjacent bars of the bar charts. The results show that compared to the negative control, vibrated phospholipid prepared as described in Example 1 significantly increased NEKa proliferation at a concentration of 4 mg/ml after 48 hours. At 72 hours, the results show that compared to the negative control, vibrated phospholipid prepared as described in Example 1 significantly increased NEKa proliferation at a concentration of 0.125 mg/ml.

Non-vibrated phospholipid prepared as described in Example 1 and dispersed in supplemented keratinocyte basal medium was used to stimulate NEKa keratinocyte cells and the data obtained from the Alamar Blue™ assay after 48 hours and 72 hours are shown in the bar charts of FIGS. 12C and 12F. In these bar charts, the first bar from the left is for the negative control (C). Non-vibrated phospholipid prepared as described in Example 1 was used at concentrations (mg/ml) of 0.125, 0.25, 0.5, 1, 2 and 4 and the results obtained for each concentration are shown in the respective adjacent bars of the bar charts. The results show that compared to the negative control, non-vibrated phospholipid prepared as described in Example 1 significantly increased NEKa proliferation at concentrations of 0.125 mg/ml and 0.5 mg/ml.

### EXAMPLE 7

The suitability of various media for use in forming a dispersion of phospholipid prepared as described in Example 1 or of DPPC was investigated. The Alamar Blue™ assay of HPMC proliferation described in Example 3 was repeated using a single well of cells for each stimulation with a positive control of FCS dispersed in the tested medium, a negative control of the tested medium, and a phospholipid dispersed in the tested medium. The results obtained after 48 hours are shown in FIGS. 13A-13J and discussed below.

The first tested medium was a 50/50 v/v mixture of Hartmanns solution (Fresenius Kabi) and supplemented M199. As a positive control, FCS in the first tested medium was used to stimulate HPMC and the data obtained is shown in the bar charts of FIGS. 13A and 13G. In these bar charts, the first bar from the left is for the negative control (C). FCS was used at concentrations (v/v %) of 0.156, 0.313, 0.625, 1.25 and 2.5 in the first tested medium and the results obtained are shown in the respective adjacent bars of the bar charts of FIGS. 13A and 13G. The results show that there was a dose dependent increased HPMC proliferation response to FCS in the first tested medium compared to the negative control. However the amount of the increase was not as great as with FCS in supplemented M199 by itself as shown in FIG. 6A.

Vibrated phospholipid prepared as described in Example 1 and dispersed in the first tested medium was used to stimulate HPMC and the data obtained is shown in the bar chart of FIG. 13B. In this bar chart, the first bar from the left is for the negative control (C). Vibrated phospholipid prepared as described in Example 1 was used at concentrations (mg/ml) of 0.156, 0.3125, 0.625, 1.25, 2.5, 5, 10, 15, 20, and 40 in the first tested medium and the results obtained are shown in the respective adjacent bars of the bar chart of FIG. 13B. The results show that the increase in proliferation response to vibrated phospholipid prepared as described in Example 1 in the first tested medium compared to the negative control was not as great as with vibrated phospholipid prepared as described in Example 1 in supplemented M199 by itself as shown in FIG. 6B.

DPPC dispersed in the first tested medium was used to stimulate HPMC and the data obtained is shown in the bar chart of FIG. 13H. In this bar chart, the first bar from the left is for the negative control (C). DPPC was used at concentrations (mg/ml) of 0.156, 0.3125, 0.625, 1.25, 2.5, 5, 10, 20 and 40 in the first tested medium and the results obtained are shown in the respective adjacent bars of the bar chart of FIG. 13H. The results show that the increase in proliferation response to DPPC in the first tested medium compared to the negative control was not as great as DPPC in supplemented M199 by itself as shown in FIG. 7B.

The second tested medium was a 50/50 v/v mixture of Ringers solution (Fresenius Kabi) and supplemented M199. As a positive control, FCS in the second tested medium was used to stimulate HPMC and the data obtained is shown in the bar chart of FIG. 13C. In this bar chart, the first bar from the left is for the negative control (C). FCS was used at concentrations (v/v %) of 0.156, 0.313, 0.625, 1.25 and 2.5 in the second tested medium and the results obtained are shown in the respective adjacent bars of the bar chart of FIG. 13C. The results show that there was a dose dependent increased HPMC proliferation response to FCS in the second tested medium compared to the negative control. However the amount of the increase was not as great as with FCS in the first tested medium as shown in FIG. 13A.

Vibrated phospholipid prepared as described in Example 1 and dispersed in the second tested medium was used to stimulate HPMC and the data obtained is shown in the bar chart of FIG. 13D. In this bar chart, the first bar from the left is for the negative control (C). Vibrated phospholipid prepared as described in Example 1 was used at concentrations (mg/ml) of 0.156, 0.3125, 0.625, 1.25, 2.5, 5, 10, 15, 20, and 40 in the second tested medium and the results obtained are shown in the respective adjacent bars of the bar chart of FIG. 13D. The results show that the increase in proliferation response to vibrated phospholipid prepared as described in Example 1 in the second tested medium compared to the negative control was better than vibrated phospholipid in the first tested medium as shown in FIG. 13B but not as great as with vibrated phospholipid in supplemented M199 by itself as shown in FIG. 6B.

The third tested medium was a 50/50 v/v mixture of saline solution (0.9% w/v sodium chloride in water) and supplemented M199. As a positive control, FCS in the third tested medium was used to stimulate HPMC and the data obtained is shown in the bar chart of FIG. 13E. In this bar chart, the first bar from the left is for the negative control (C). FCS was used at concentrations (v/v %) of 0.156, 0.313, 0.625, 1.25 and 2.5 in the third tested medium and the results obtained are shown in the respective adjacent bars of the bar chart of FIG. 13E. The results show that there was a dose dependent increased HPMC proliferation response to FCS in the third tested medium compared to the negative control. However the amount of the increase was not as great as with FCS in the first tested medium as shown in FIG. 13A.

Vibrated phospholipid prepared as described in Example 1 and dispersed in the third tested medium was used to stimulate HPMC and the data obtained is shown in the bar chart of FIG. 13F. In this bar chart, the first bar from the left is for the negative control (C). Vibrated phospholipid prepared as described in Example 1 was used at concentrations (mg/ml) of 0.156, 0.3125, 0.625, 1.25, 2.5, 5, 10, 15, 20, and 40 in the third tested medium and the results obtained are shown in the respective adjacent bars of the bar chart of FIG. 13F. The results show that the increase in proliferation in response to vibrated phospholipid in the third tested medium compared to the negative control was not as great as vibrated phospholipid in the first tested medium as shown in FIG. 13B.

The fourth tested medium was a 90/10 v/v mixture of Hartmann's solution and supplemented M199. As a positive control, FCS in the fourth tested medium was used to stimulate HPMC and the data obtained is shown in the bar chart of FIG. 13I. In this bar chart, the first bar from the left is for the negative control (C). FCS was used at concentrations (v/v %) of 0.156, 0.313, 0.625, 1.25 and 2.5 in the fourth tested medium and the results obtained are shown in the respective adjacent bars of the bar chart of FIG. 13I. The results show that FCS in the fourth tested medium did not increase HPMC proliferation compared to the negative control. Accordingly it is clear that the fourth tested medium was not as effective as supplemented M199 by itself from a comparison of the results shown in FIG. 13I with those of FIG. 6A.

Vibrated phospholipid prepared as described in Example 1 and dispersed in the fourth tested medium was used to stimulate HPMC and the data obtained is shown in the bar chart of FIG. 13J. In this bar chart, the first bar from the left is for the negative control (C). Vibrated phospholipid prepared as described in Example 1 was used at concentrations (mg/ml) of 0.156, 0.3125, 0.625, 1.25, 2.5, 5, 10, 15, 20, and 40 in the fourth tested medium and the results obtained are shown in the respective adjacent bars of the bar chart of FIG. 13J. The results show that the increase in proliferation in response to vibrated phospholipid in the fourth tested medium compared to the negative control was not as great as with vibrated phospholipid in supplemented M199 by itself as shown in FIG. 6B.

### EXAMPLE 8

In this Example, the properties of dispersions formed by a phospholipid prepared as described in Example 1 were investigated at different temperatures and in different media.

In a first experiment, 0.5 mg/ml of a phospholipid prepared as described in Example 1 was dispersed in M199 medium at room temperature. After 30 minutes, the micrograph shown in FIG. 17A was taken using an Axiovert 100M inverted microscope with a 5× objective (Carl Zeiss, Oberkochen, Germany) such that the final magnification in the image shown is 50×. This micrograph shows that the dispersion of the phospholipid according to the invention comprises discrete crystals of phospholipid.

In a second experiment, 0.5 mg/ml of a phospholipid prepared as described in Example 1 was dispersed in M199 medium at 37° C. After 30 minutes, the micrograph shown in FIG. 17B was taken in the same way as for FIG. 17B. This micrograph shows a fine micellar dispersion of the phospholipid according to the invention which has substantially no agglomerations of micelles.

In a third experiment, 1 mg/ml of a phospholipid prepared as described in Example 1 was dispersed at room temperature in Ringers lactate, water for injection and saline and each dispersion was placed in a respective labelled vial. After 30 minutes had elapsed, the photograph of the vials shown in FIG. 18 was taken. This photograph shows that the dispersion of the phospholipid according to the invention in water for injection was so fine that it was not visible to the naked eye. Thus the dispersion only has slight cloudiness and is translucent. The dispersions of the phospholipid according to the invention in Ringers lactate and saline are not as fine as the dispersion of the phospholipid according to the invention in water for injection. These two dispersions are cloudy but it is clear that the dispersions lack visible or macroscopic agglomerations of micelles.

### EXAMPLE 9

The phospholipid prepared as described in Example 1 has been shown in Example 2 to promote the re-epithelialisation of monolayers of mesothelial cells and keratinocytes damaged by scratch wounding. This it achieves partly through effects on cell proliferation and cell migration (Yung, S. et al; 1998; “Response of the human peritoneal mesothelial cell to injury: an in vitro model of peritoneal wound healing.” Kidney international 54:2160-2169; and Yung, S. et al 2000 “Induction of hyaluronan metabolism after mechanical injury of human peritoneal mesothelial cells in vitro”; Kidney international 58:1953-1962).

In order to establish whether the phospholipid prepared as described in Example 1 has similar effects on repair processes relevant to tendon injury experiments will be performed with the synovial cells lining the tendon sheath or cells derived from the tendon or epitenon (Lundborg, G. et al; 1978; “Experimental intrinsic healing of flexor tendons based upon synovial fluid nutrition” The Journal of hand surgery 3:21-31; Manske, P. R. et al; 1984; “Histologic evidence of intrinsic flexor tendon repair in various experimental animals. An in vitro study.” Clinical orthopaedics and related research: 297-304; Gelberman, R. H. et al; 1984; “Flexor tendon repair in vitro: a comparative histologic study of the rabbit, chicken, dog, and monkey”; Journal of orthopaedic research 2:39-48). The tendon complex is composed of cells from epitenon (outer surface of the tendon), endotenon (within the tendon) and the synovial cells lining the tendon sheath. There is evidence that the rates of cell proliferation vary considerably between these three cell types in response to growth factors (Khan, U. et al; 1998; “Differences in proliferative rate and collagen lattice contraction between endotenon and synovial fibroblasts.” The Journal of hand surgery 23:266-273).

### Methods:

In order to assess the impact of the phospholipid prepared as described in Example 1 on cell proliferation and repair following wounding tendon or sheath-derived cells (sheath, epitenon and endotenon) will be established in monolayer culture to confluence or sub-confluence (Vater, C. A. et al; 1979; “Inhibitor of human collagenase from cultures of human tendon.” The Journal of biological chemistry 254:3045-3053; Becker, H. et al; 1981; “Intrinsic tendon cell proliferation in tissue culture.” The Journal of hand surgery 6:616-619).

In confluent cultures, the cells will be scratch wounded as previously described (Yung et al. 1998, 2000) with a metal rod. Wound closure will be monitored in the presence or absence of increasing doses of the phospholipid prepared as described in Example 1 (0.1-4 mg/ml) for periods up to 7 days by time-lapse videomicroscopy (Morgan, L. W. et al; 2003; “Glucose degradation products (GDP) retard remesothelialization independently of D-glucose concentration” Kidney international 64:1854-1866).

In sub-confluent cells, tendon-derived cell proliferation will be assessed over a 72 hour period, following treatment by the phospholipid prepared as described in Example 1 or control treatment, by Alamar blue cell proliferation assay (Squatrito, R. C. et al; 1995; “Comparison of a novel redox dye cell growth assay to the ATP bioluminescence assay” Gynecologic oncology 58:101-105).

### EXAMPLE 10

ALEC® (Britannia Pharmaceuticals Ltd) has previously been shown to reduce adhesion formation in a mouse model, it is likely therefore that the phospholipid prepared as described in Example 1 may exert similar protective mechanisms in reducing tendon adhesions following surgical repair. Following tendon injury it is believed that adhesion formation involves the increased deposition of collagen and other extracellular matrix components (e.g. collagen and fibronectin) resulting in scar formation. Previous work has demonstrated that changes in TGF-β mRNA expression (a potent modulator of extracellular matrix turnover) can occur in both the endotenon and epitenon and tendon sheath cells in vitro (Chang, J. et al; “Gene expression of transforming growth factor beta-1 in rabbit zone II flexor tendon wound healing: evidence for dual mechanisms of repair.” Plast Reconstr Surg 1997; 100: 937-994).

### Methods:

In order to assess the impact of Phospholipid prepared as described in Example 1 on tendon cell extracellular matrix production tendon cells (sheath, epitenon and endotenon) will be established in monolayer cultures to sub-confluence (Vater, C. A. et al; 1979; “Inhibitor of human collagenase from cultures of human tendon.” The Journal of biological chemistry 254:3045-3053; Becker, H. et al; 1981; “Intrinsic tendon cell proliferation in tissue culture” The Journal of hand surgery 6:616-619).

In sub-confluent tendon-derived cells both constitutive and cytokine-driven (e.g. interleukin-1) growth factor (TGF-β) and extracellular matrix (collagen I/III and fibronectin) mRNA expression will be assessed by quantitative PCR (qPCR) using the Applied Biosystems (ABI) 9700 multi-analyzer using commercially available validated primer sets. These experiments will be performed over a 72 hour period in the presence or absence of increasing doses of a phospholipid prepared as described in Example 1 (0.1-4 mg/ml).

### EXAMPLE 11

Adhesion formation following repair of tendon injury represent a major clinical problem. These can occur between closely opposed tendons or between the tendon and the tendon sheath. Since tendons injuries do not heal through regenerative process but via the formation of scar tissue tendon repairs often have compromised biomechanical properties that may result in impaired function (gliding of tendons and in limited mobility) or further damage (ruptures). Attempts to reduce tendon adhesion formation have been largely unsuccessful and there are limited therapies available in the market.

In order to establish whether a phospholipid prepared as described in Example 1 has the potential to improve tendon healing and function following injury and surgical repair, an established and validated in vivo model will be used. It is now well established that the rabbit forepaw flexor tendon repair model is the model of choice for quantifying tendon healing and function following injury (Lundborg, G. et al; 1985; “Intrinsic tendon healing. A new experimental model” Scandinavian journal of plastic and reconstructive surgery 19:113-117; Greenwald, D. et al; 1991; “Biomechanical analysis of intrinsic tendon healing in vitro and the effects of vitamins A and E” Plastic and reconstructive surgery 87:925-930; discussion 931-922).

### Methods:

Surgery will be conducted on digits 2 and 4 of the right forepaw of 8 rabbits in each treatment group, with injured untreated tendons acting as adhesion controls. An incision will be made proximal to the metacarpophalangeal joints, avoiding direct injury to the synovial sheath. The intrasynovial portion of the flexor digitorum profundus tendon will be accessed by retraction proximally and a partial tenotomy will be made. The tendon injury will be coated with the phospholipid prepared as described in Example 1 and the tendon returned to the intra-synovial position. Digits will be harvested at two weeks and the tendon-synovial adhesion complex will be carefully dissected out.

### Assessment of Tensile Strength

Untreated digits are used to determine control adhesion strength. The treated injured digits are tested to investigate the effect of the phospholipid prepared as described in Example 1 on modifying adhesion strength.

The use of a tensile testing machine is well established for assessing the biomechanics of tendon repair and adhesion formation (He Q et al., “Repair of flexor tendon defects of rabbit with tissue engineering method” Chin J Traumatol 2002; 5: 200-8; Jones M E et al, “The role of human-derived fibrin sealant in the reduction of postoperative flexor tendon adhesion formation in rabbits” J Hand Surg (Br) 2002; 27: 278-82). The digital claw is held using a clamp and the FDP Tendon is transfixed with a silk 2/0 suture connected to a tensile testing machine (Zwick Roell Group, Ulm, Germany). Pullouts are carried out at constant speed (2.5 mm/min) to determine the force required to draw the tendon from its sheath with the tendon transected at its insertion just before assessment.

### EXAMPLE 12

The aim of this Example was to assess if a phospholipid prepared according to Example 1 altered proliferation in fibroblasts derived from tendons, had an effect on extra-cellular matrix synthesis of fibroblasts derived from tendons, altered gene transcription and prevented the formation of adhesions in an in vivo model.

Flexor tendon injury is a common injury with nearly one-third of a million digital flexor tendon injuries per year in the United States (Pennisi, 2002). Despite current optimum surgical technique and compliance with early post-repair motion protocols poor results still occur in up to 15-20% of repaired lacerated intrasynovial digital flexor tendons in the traumatised hand. Poor results are frequently the result of disabling adhesions if the injury is in the region of the digital synovial sheaths (Potenza 1963; Matthews and Richards 1976; Manske, 1988; Khan et al. 1996). Half of these require additional surgery in the form of flexor tenolysis (Strickland 2000). A two-stage tendon reconstruction requires a year off work to complete the surgery and rehabilitation and 28% are rated as having a poor outcome in staged tendon reconstruction.

Tendons do not heal through a regenerative process: instead healing occurs via the formation of a fibrotic scar (Gelberman et al, 1985a, 1986, 1985b; Speer et al, 1985; Hatano et al, 2000). This repair has inferior biomechanical properties leading to compromised function. Further, adhesions between the tendon and sheath can impair the gliding mechanisms of tendons and result in limited mobility (Manske et al, 1985). Though new surgical techniques and rehabilitation have improved results, adhesions still remain a significant problem (Gelberman et al, 1985a, Porat et al, 1980; Speer et al, 1985; Manske et al, 1985; Thomas et al, 1986; Nyska et al, 1987; Beredjiklian, 2003). Attempts to modulate adhesion formation to date have proven unsuccessful and have concentrated on manipulation of the wound-healing process by physical or pharmacological means and can result in reduced adhesion formation but with a corresponding increase in rupture rate (e.g. ADCON; Golash et al, 2003).

Tendon healing has three sequential phases of repair: inflammation, fibroblastic and remodelling. There have been a number of studies who have proposed two mechanisms for tendon repair: extrinsic whereby the tendon cells and inflammatory cells migrate from the periphery into the repair site to promote healing or the intrinsic whereby cells from within the tendon (endotenon) migrate into the repair site (Manske et al, 1985; Gelberman et al, 1986), with extrinsic repair associated with adhesion formation. Though recent work has suggested that tendon repair is through a combination of both extrinsic and intrinsic repair. It has been well established that migrating fibroblasts are responsible for adhesion formation and that fibroblast chemotaxis and adherence to the substratum in the days after injury and during repair appears to be related directly to fibronectin secretion (Gelberman et al, 1991). Others have suggested that fibronectin and the TGFβ1 RI are up regulated in tendons with heavy adhesion formation (Ngo et al, 203).

Uninjured tendons are composed of dense regularly organised collagen fibres which allow connection from bone to muscle. The tendon structure is composed longitudinally arranged collagen fibres, with the uninjured tendon being composed of 60-85% collagen type I and only 0-10% being composed of collagen type III (dry weight). If the ratio of the collagen type or its cross-linking is altered the tendons are less resistant to loading or too much extra-cellular matrix is produced then there will be adhesion formation.

### Materials and Methods:

Epitenon (Surface), endotenon (core) and sheath derived tendon cells have been shown to have different proliferation rates in response to growth factors (Khan et al, 1998), blocking antibodies (Zhang et al, 2004), extra cellular matrix gene transcription (Berguland et al, 2006), collagen gel contraction (Ragoowansi et al, 2003), and fibronectin secretion (Gelberman et al, 1991). Therefore all three-cell types were established and cultured as separate cell strains.

Tendon derived fibroblasts were cultured in Dulbecco's Modified Eagles Medium (DMEM) with 10% fetal calf serum, 2 mM glutamine, 100 U/ml penicillin and 100 mg/ml streptomycin (NGM).

### Preparation of Phospholipid

A 5 mg/ml solution of the phospholipid prepared according to Example 1 and referred to in this example as “Pumactant” was dissolved in serum free media in a sterile 100 ml bottle incubated at 37° C. and then diluted further to a concentration of 1 mg/ml.

### Preparation of TGF-β1

TGF-β1 was diluted as per manufactures instructions at a stock concentration of 2 ng/μl. Concentration used 2 ng/μl (previously optimised).

### Preparation of Fetal Calf Serum

Concentrations of 5% (v/v), 2.5%, 1.25%, 0.6%, 0.3% fetal calf serum (FCS) was diluted in serum free media.

### Proliferation Assay:

Fibroblasts derived from tendons were seeded at 5×10³/well into a 96 microtitre plate (Greiner) and allowed to attach overnight in minimum media (DMEM supplemented with 0.4% FCS) to maintain cells in a healthy but quiescent state. After 24 hrs, the media was aspirated and replaced by test media (minimum media with concentrations of pumactant; or different concentrations of fetal calf serum; or different concentrations of TGF-β1) and cultured for 24, 48 and 72 hours. Cell number was assessed using WST-1 assay (Roche Products Ltd). Test media was removed and the wells washed twice with minimal media, minimal media was replaced and 10 μl WST-1 solution was added to each well. The cells were incubated for 1 hour in standard tissue culture conditions. The absorbance, which related to the number of viable cells converting the reagent to coloured formazan crystals, was read at 450 nm on a Bio-Rad Plate reader. Experiments were performed in triplicate for each cell strain.

### Adhesion Assay:

Nunc maxabsorb 96 well culture plate was used, with wells coated with either Collagen type I, Collagen type III, fibronectin or left un-coated. Wells were set up in triplicate. Cells were removed from plastic flasks using Accutase and re-suspended into NGM. 2×10⁵ cells/ml were required. Cells were incubated with treatment (pumactant 1 mg/ml) at 37° C. for 30 mins in suspension. 100 μl cell and treatment solution were plated into coated wells. The cells/treatment solution are incubated for 2 hours at 37° C. in humidified 5% CO₂. The treatment is aspirated off and the wells gently washed with PBS. Finally the cells are fixed and stained with crystal violet solution (0.5% crystal violet, 5% formol saline, 50% ethanol, 0.85% NaCl). Crystal violet is removed and the wells carefully washed with Phosphate Buffered Saline. The bound crystal violet was solubilised with 33% acetic acid and the plate was read in a Bio-Rad Plate reader at 595 nm.

### Extra-Cellular Matrix Production:

Sircol assay (Biocolor Ltd), was performed as described previously (Rolfe et al, 2007). Fibroblasts derived from tendons were seeded at 10⁶ cells/25 cm² flask and allowed to attach and spread overnight in NGM. Cells were washed with PBS twice and media replaced with DMEM with 5% fetal calf serum+/−pumactant (1 mg/ml) or +/−TGF-β1 (2 ng/ml). Conditioned media was then collected. Cells were trypsininsed and counted, to correct the subsequent collagen quantification for cell number. Due to the binding of pumactant to the Sirus red dye, the conditioned media containing the pumactant was centrifuged at 10,000 g for 5 mins (following optimisation, data not shown). 200 μl conditioned media was incubated with 1 ml Sircol dye for 30 mins. Tubes were then centrifuged at 10,000 g for 10 mins to pellet the precipitated collagen-dye complex.

Supernatant was removed and 1 ml alkali reagent was added to the collagen pellet. The dissolved collagen and dye solution was then aliquoted (200 μl/well) in triplicate into a 96 well plate along with collagen standards. The solutions were then read at 540 nm. All experiments were repeated ×3 and each experiment run in triplicate. Results were standardised for cell number.

### QRT-PCR:

Cells were placed in serum free media for 24 hrs prior to stimulation with pumactant or TGF-β1; viability was assessed using Trypan blue (Sigma; data not shown). Quiescent cells were treated with pumactant (1 mg/ml) for 0-24 hrs or TGF-β1 for 0-24 h ours (positive control). Total RNA was isolated from treated and untreated samples using Trizol (Gibco). Quant-iT™ Ribogreen® RNA kit (Invitrogen) was used to determine RNA concentration. First strand cDNA was synthesized using the StrataScript® first-strand synthesis system (Stratagene™) with oligonucleotide dTs (0.5 μg/μl) according to manufacturer's instructions.

### Quantitative Real-Time PCR

Two μl of the first strand cDNA product was used for amplification in triplicate in a 25 μl reaction solution containing 12.5 μl of SYBR Green PCR Master Mix (Stratagene™, La Jolla, Calif.) and 10 pM of each primer as per manufacturer's instructions. Primer sequences are identified in Table 1. The primer sequences are also listed in the sequence listing which is attached to the specification of the present application and made a part thereof.

TABLE 1

| | | SEQ | |
|---|---|---|---|
| | Primer | ID | |
| Gene | type | NO. | Sequence |
| Collagen | F | 1 | 5′ CAAACCTCTT |
| III: | | | CCTGAAGCC 3′ |
| | R | 2 | 5′ ATTATAGCAC |
| | | | CATTGAGAC 3′ |
| tPA | F | 3 | 5′ GAGGCTCACGTCCG |
| | | | GCTGTACCCCTCCA 3′ |
| | R | 4 | 5′ TCCTTCTGCCCACAG |
| | | | CCCAGCCCCCAG C 3′ |
| Collagen I | F | 5 | 5′ TTCTTGGTGC |
| | | | TCCTGGCATTC 3′ |
| | R | 6 | 5′ GCAATCCGTTG |
| | | | TGTCCCTTTATG 3′ |
| PAI-1 | F | 7 | 5′ GGATTTGG |
| | | | CCGCATTG 3′ |
| | R | 8 | 5′ CAACATCCACTT |
| | | | TGCCAGAGTTAA 3′ |
| Fibronectin | F | 9 | 5′ TCGGGAGGAAGA |
| | | | AGACAGATGAGC 3′ |
| | R | 10 | 5′ ACCACTGCCAA |
| | | | AGCCTAAGCAC 3′ |
| GAPDH | F | 11 | 5′ GGATTTGG |
| | | | CCGCATTGG 3′ |
| | R | 12 | 5′ CAACATCCACTT |
| | | | TGCCAGAGTTAA 3′ |
| ACTB | F | 13 | 5′ GCTCGTCGTC |
| | | | GACAACGGCTG 3′ |
| | R | 14 | 5′ CAAACATGATCT |
| | | | GGGTCATCTTCTC 3′ |

The PCR reaction was performed on a MX3000P (Stratagene, La Jolla, Calif.). The PCR program consisted of an initial denaturation where the reaction was incubated for 10 min at 95° C., the second step the DNA was amplified for 40 cycles of 30 sec at 95° C. annealing of primers for 1 min at 60° C. and an extension at 72° C. for 30 sec. Dissociation curve was performed to ensure no primer-dimers were present at the end of each PCR run.

### In Vivo Assay

Chang et al (2000) have demonstrated that the rabbit flexor tendon repair model is useful for quantifying tendon scar formation on the basis of degrees of flexion between proximal and distal phalanges.

All animal care complied with the ‘UK Home office Guide for the Care and the use of Laboratory Animals’ 1996. Surgery was conducted as described previously (Branford et al) on digits 2 and 4 of the right forepaw of 16 New Zealand White rabbits (NZW) under general anaesthesia (Fentanyl citrate and fluanisone and isoflurane/O₂). The operative site was shaved and prepared with chlorexidine. A ‘V’ incision proximal to the metacarpophalangeal joints gave access to the flexor digitorum profundus (FDP) tendons while avoiding direct injury to the synovial sheath. The flexor digitorium superficaialis tendon was excised, limiting adhesions to those formed between the FDP tendon and the sheath. The FDP tendon was retracted exposing the intrasynovial tendon. A half thickness and 5 mm long injury was created (Jones et al, 2002) with a scalpel (this technique has been described in a number of publications (Kakar et al, 1998; Akali et al, 1999; Khan et al, 2000). On extending the digits the injury was returned to the intrasynovial position, tendons were not sutured and were immobilised (by proximal transaction).

NZW were randomised to those treated with pumactant (10 mg/ml dissolved in sterile water) or left untreated. The tendon sheath was infiltrated with the treatment (0.5 mls of 10 mg/ml pumactant per sheath) using a 24 gauge cannula with the needle removed.

Remaining pumactant was placed under the flap of skin. The skin was closed using 4/0 nylon sutures cleaned and an antibiotic powder applied topically (Aureomycin). Animals were allowed to move around and then culled at 14 days.

Digits two and four of the left forepaw were harvested as un-operated controls. Digits were randomised to i. Mechanical assessments or ii. quantitative histological assessments.

### Mechanical Evaluation:

The relationship between tendon/adhesion ultimate tensile strength depends on composition and collagen fibril length and mechanical behaviour. The use of a tensile testing machine is well established in the literature for assessing the biomechanics of tendon repair and adhesion formation (He et al, 2002; Jones et al, 2002; Momose et al, 2001, Branford et al, 2007).

Forepaws were freshly assessed. Prior to testing the V shaped wounds were reopened and the proximal free ends of each treated tendon were identified. A distal transection was made over the insertion of the FDP into the distal phalanx. This allows the tendon to lie freely within the sheath, disconnected at either end such that a force applied proximally would solely measure the strength of any adhesion present between the sheath and the tendon. Each digit claw was held using a clamp and the proximal FDP tendon transfixed to a tensile testing machine (Mecmesin). The proximal tendon was then pulled from its sheath (at a rate of 5 mm/min) until the adhesion failed. The peak force was measured in Newtons.

### Histological Analysis:

Digits were fixed in formalin, decalcified in EDTA (ethylenediamine tetraacetic acid) and paraffin embedded. Longitudinal sections were cut at standardised depths to assess throughout the tendon any adhesion formation. The sections were then stained with either haematoxylin and eosin or Masson Trichome (to determine the structure of the collagen). The presence of adhesions on microscopic examination as a characteristic densely cellular band connecting the tendon to surrounding tissues was recoded and the adhesions were then scored as described by Tang et al (1996), as shown in Table 2 below.

TABLE 2

| Points | Features of Adhesions |
|---|---|
| | Quantity |
| 0 | No apparent adhesions |
| 1 | A number of scattered filaments |
| 2 | A large number of filaments |
| 3 | Countless filaments |
| | Quality |
| 0 | No apparent adhesions |
| 1 | Regular, elongated, fine, filamentous |
| 2 | Irregular, mixed, shortened, filamentous |
| 3 | Dense, not filamentous |
| | Grading of adhesions |
| 0 | No adhesions |
| 2 | Slight adhesions |
| 3, 4 | Moderate adhesions |
| 5, 6 | Severe adhesions |

### Data Analysis:

The relative expression software tool (REST©) was used for the calculation of relative expression levels in real-time PCR (Pfaffl et al, 2002). This mathematical model is based on the PCR efficiencies and the mean crossing point deviation between the treated and untreated cells. The target gene expression was normalized with two housekeeping genes: GAPDH and ACTB. Statistical analysis of group differences was performed by Pair Wise Fixed Reallocation Randomization Test©, implemented in the REST-XL software (Pfaffl et al, 2002) and the coefficient of variance was given. Sigma Plot was used to illustrate the logarithmic graphs.

Statistical significance was analyzed using a Student t-test where appropriate. A p value of <0.05 was considered statistically significant.

### Results

### Proliferation:

Tendon Sheath: Cells derived from the tendon sheath cultured in 0.25 mg/ml of pumactant showed a significant reduction in cell number at 24 hours (p=0.04; FIG. 19). Though the rest of the concentrations saw an increase in cell number none of the concentrations showed a statistical significance compared to cells grown solely in minimal media. There was no statistical significance in sheath cells cultured in various concentrations of either TGF-β1 or FCS (data not shown).

Tendon derived sheath cells cultured in pumactant showed no statistical significance for any concentration at 48 hours (FIG. 20A). However, tendon sheath cells at 48 hours (FIG. 20B) showed a statistically significant increase in proliferation in various concentrations of FCS: 0.6% (p=0.025), 1.25% (p=0.03), 2.5% (p=0.003) and 5% (p=0.01) compared to cells grown in serum free media. Cells derived from the tendon sheath showed no statistical significance when cultured in any concentration of TGF-β1 at 48 hours (data not shown).

Tendon sheath cells at 72 hours (FIG. 21A) cultured in pumactant showed a statistical significant increase at both 4 mg/ml (p=0.022) and 2 mg/ml (p=0.018) compared to cells grown solely in minimal media. All concentrations of fetal calf serum showed a statistical increase compared to cells grown in serum free media (FIG. 21B).

Tendon surface cells: Cells derived from the surface of the tendon at 24 hours (FIG. 22A) showed an increase in proliferation at 2 and 4 mg/ml when cultured in pumactant but this did not reach statistical significance. Tendon surface cells showed a statistical significant increase at 0.6% (p=0.027) and 2.5% (p=0.001; FIG. 22B) fetal calf serum compared to cells grown in serum free media, whereas cells cultured in TGF-β1 showed no statistical significant difference at any concentration.

At 48 hours tendon surface cells showed a statistically significant increase when cultured with 2 mg/ml of Pumactant compared to cells grown in minimal media (FIG. 23A). All concentrations of fetal calf serum, with the exception of 0.6% showed a statistically significant increase compared to cells cultured in serum free media (FIG. 23B). No concentrations of TGF-β1 showed any statistical increase at 48 hours (data not shown).

Tendon surface cells at 72 hours (FIG. 24A) showed an increase in proliferation when cultured in 2 and 4 mg/ml of pumactant but this did not reach statistical significance. Tendon surface cells showed a statistically significant increase in proliferation at 2.5% (p=0.03) and 5% (p=0.04; FIG. 24B) fetal calf serum compared to cells grown solely in serum free media. Further tendon surface cells cultured in 2 ng/ml of TGF-β1 showed a statistically significant increase in proliferation (p=0.003; FIG. 24C).

Tendon core cells: Core cells cultured in pumactant showed an increase in proliferation from 0.5 mg/ml-4 mg/ml but no concentration showed a statistical significant increase compared to cells grown in minimal media (FIG. 25). Further there was no statistical increase in proliferation for any percentage of FCS or concentration of TGF-β1 (data not shown). However cells derived from tendon core cultured for 48 hours in pumactant showed a statistical significant increase in cell proliferation at 0.5 mg/ml (p=0.049; FIG. 26) compared to those cultured in minimal media. There was no other significance for any concentration or treatment. Core derived cells at 72 hours showed no statistical significance for pumactant (FIG. 27) or concentrations of fetal calf serum of TGF-β1 (data not shown).

### Gene Transcription:

All genes examined were expressed at detectable levels in all cell types with or without treatment.

### Surface Derived Cells:

Cells derived from the surface of the tendon where cultured with TGF-β1 (positive control). Culturing with TGF-β1 caused the cells to show an up regulation of Collagen type I gene transcription which was statistically significant at 24 and 48 hours (p<0.05; FIG. 28). Conversely collagen type III showed a statistically significant down regulation at 1 hour, 2, 6, 16 and 48 hours (p<0.05). Whereas fibronectin and PAI-1 both showed an up regulation in response to TGF-β1, which was statistically significant at 24 hours for fibronectin (p=0.03) and 4 hours for PAI-1 (p=0.026).

No gene studied showed any statistical change over the time course in response to Pumactant (FIG. 29).

### Sheath Derived Cells:

TGF-β1 caused cells derived from the sheath to up regulate of Collagen type I gene transcription over the time course though this did not reach statistical significance. Whereas Collagen type III showed an up regulation, which was statistically significant at 6 hours, 8 hours (p=0.03; FIG. 30), 16 and 24 hours (p<0.05). Fibronectin and PAI-1 showed no statistical changes over the whole time course. Whereas tPA showed a down regulation of gene transcription which was statistically significant at 2 hrs (p=0.013), 4 hours and 24 hours (p=0.001).

Cells originating from the sheath cultured in pumactant showed a down regulation of Collagen type I at the start of the time course which from 16 h (FIG. 31) showed an up regulation though at no point did this reach statistical significance. Collagen type III showed an up regulation at all time points except at 48 hours though again this failed to reach statistical significance. Fibronectin gene transcription was up regulated in response to pumactant but failed to reach statistical significance. PAI-1 showed a statistical down regulation of gene transcription at 1 h (p=0.001) whereas tPA showed a down regulation through at the time course but this did not reach statistical significance.

### Core Derived Cells:

Core derived cells showed no statistical change when cultured with TGF-β1 (2 ng/ml) in collagen type I gene transcription over the 48-hour time course. Whereas collagen type III gene transcription showed a statistically significant down regulation in the early stages of the time course 2, 6 and 8 hours (p=0.03; FIG. 32), however at the later time points showed an up regulation which however was not statistically significant. Fibronection gene transcription showed at 6 h a statistically significant down regulation (p=0.024), which was later, reversed with a statistically significant up regulation at 24 and 48 hours (p<0.05). Plasminogen activator inhibitor-1 (PAI-1) showed an up regulation of gene transcription at 2 and 4 hours but this was only statistically significant at 2 hours (p=0.001). Whereas tPA showed a statistically significant down regulation in gene transcription at 1, 2, 6, 8 and 24 hrs 0.01) and 16 hours (p=0.002).

Core derived cells cultured in pumactant showed a down regulation of collagen type I gene transcription though at no time point did this reach statistical significance when cultured with 1 mg/ml pumactant. However collagen type III showed a statistically significant up regulation at 24 and 48 hours (p=0.025; FIG. 33). Fibronectin showed no statistical change over the time course. Whereas PAI-1 showed a statistically significant up regulation of gene transcription in response to pumactant at the early time points 1 and 2 hours (p=0.001) and then later showed a statistically significant down regulation at 6, 8 and 16 hours (p=0.001). tPA gene transcription showed no significant change over the entire time course.

### Adhesion Assay:

Tendon derived fibroblasts showed no statistical significant difference in their adhesion to any of the matrices studied whether they were cultured with or without pumactant (1 mg/ml; FIG. 34).

### Extra Cellular Matrix Production:

Western blotting was performed for Collagen type I using a variety of commercially available primary antibodies (to detect rabbit Collagen type I) but though the loading controls worked consistently none of the commercially available antibodies produced any bands even when examining protein lysates from cells cultured with TGF-β1. Western blotting was therefore abandoned.

The Sircol collagen assay is a quantitative dye-binding method designed for the analysis of soluble collagens released into culture medium by mammalian cells in vitro. Mammalian collagens Types I to XIV are detected but the dye does not discriminate between them. The dye reagent binds specifically to the [Gly-X-Y] helical structure found in all mammalian collagens. Pumactant had no effect on the production of soluble collagen for any of the tendon cell types when compared to either cells cultured in media alone (with 5% FCS; FIG. 35) or those with TGF-β1.

### In Vivo Assay:

### Mechanical Assessment

The raw data from the tensile testing machine (FIG. 36) demonstrates an observable difference between the operated untreated (OUT) and un-operated untreated groups (UOUT). These two groups represent the positive and negative control groups for this study respectively. Essentially, when a tendon is operated on adhesions will form around the site of injury as found in the OUT-group (because the rabbits paw has been immobilised). A mean force of 3.78N (FIG. 36) is a typical finding for this group. If there has been no tendon injury there should be no adhesion formation as represented by the UOUT-group. However, in this latter group we do occasionally find small increases in load (0.2-0.6N) as displacement increases.

Comparing the raw data from the operated pumactant group with the un-operated untreated (UOUT-group; FIG. 37) there is only a minor difference in the force required to pullout the tendon from the sheath. The forces are small 0.4N and 0.6N and it is likely that these again are due to vinculae rather than any adhesions formed.

The operated untreated group showed a pull out of 2.8±1.1, the un-operated showed a pull out of 0.398±0.2, while the pumactant group (operated and treated with pumactant) 0.8±0.8. The operated but untreated group required a statistically significant increase in force compared to both the un-operated and pumactant treated groups (p<0.0001; FIG. 38). There was a statistically significant reduction in force required to break adhesions when the pumactant treated group is compared to the operated untreated group (p=0.0012).

### Histological Assessment

All un-operated and untreated (UOUT) samples showed, as expected, no evidence of adhesion formation on macroscopic or microscopic examination. Masson's Trichrome staining demonstrated a more organised collagen fibril formation in the un-operated compared to the operated tendon groups. It also showed new extracellular matrix (ECM) deposition as demonstrated by red staining (FIG. 39). Operated untreated (OUT) tendons frequently showed a dense and irregular adhesion pattern between the tendon and tendon sheath (FIG. 40), whereas pumactant treated tendons occasionally showed a few scattered filaments of adhesion tissue or sometimes an adhesion could not be observed (FIG. 40). Operated untreated (OUT) samples showed an adhesion score based on Tang et al (1996) ranging between 4-6 (FIG. 41) (Mean=5.13±0.84) whereas tendons treated with pumactant showed a wider range of scores 0-5 but were lower on average (Mean=1.88±2.10). There was a statistically significant difference (p=0.0012; twenty-seven) between these two groups. These results show that pumactant can reduce the histological appearance of adhesion formation.

### Summary of Results:

### Proliferation:

Pumactant showed a variable response in increasing proliferation from the cells derived from different areas of the tendon-sheath complex. With surface derived cells showing a statistically significant increase at 2 mg/ml at 48 hours, whereas core showed a statistically significant increase at the same time but at 0.5 mg/ml. Sheath showed a statistically significant increase at 72 hours at both 2 and 4 mg/ml.

### Gene Transcription:

Surface and sheath showed no statistical significant change in gene expression for any of the genes studied, though cells derived from the core (endotenon) showed a statistically significant increase for collagen type III at 24 and 48 hours and for PAI-1 at 1 and 2 hours.

### Adhesion Assay and Collagen Production:

No statistical significance was identified in any cell type for either method.

### In Vivo:

Pumactant showed a statistically significant reduction in both the histology/presence of adhesions and the force required to pull out the tendon.

### DISCUSSION AND CONCLUSION

Pumactant did increase proliferation in all three cell types but this was neither consistent for dosage nor time and the variability between cell strains was large. However both surface and sheath derived cells showed an increase in proliferation in response to various concentrations of fetal calf serum at 48 and 72 hours, and both these cell types also showed an increase in proliferation to TGF-β1 at 72 hours. Core derived cells showed no statistically significant increase with either fetal calf serum or TGF-β81, but others (Khan et al, 1998; Kakar et al, 1998) have also shown that the endotenon (core derived) cells show a reduced proliferative rate both in vivo and in vitro.

Pumactant appears to have little effect on gene transcription and where it does increase gene transcription this was only demonstrated in core cells. Unlike the equivalent cells treated with TGF-β1 which showed an increase in transcription for a number of genes for all three cell types. Therefore it appears that pumactant does not appear to alter gene transcription for genes involved in tendon healing (collagen type I, Collagen type III) or adhesion formation (fibronectin, PAI-I). Further pumactant caused no statistical increase in soluble collagens, however this is collagen released into the conditioned cell culture media and does not include collagens plated on to the plastic.

Pumactant appears to have no effect on either cell adhesion to various matrices; preventing tendon derived cells from adhering to fibronectin (fibronectin is deposited early in tendon healing (Williams et al, 1984) which is thought to form a bridge between tendon and sheath. Fu et al (2005) further found that preventing tendon derived cells from adhering to fibronectin may increase collagen type I production and therefore aid in tendon healing.

Pumactant had a significant effect on the in vivo model not only reducing the amount of force required to pull out the tendon from the sheath compared to tendons which were operated on but were not treated with pumactant. But also the histological scores were statistically better in the pumactant treated group. Pumactant appears to have very little biological effect on tendon derived cells, but did improve the outcome on the in vivo model.

### REFERENCES

Akasak T et al. Hyaluronic acid diminishes the resistance to excursion after tendon flexor tendon repair: an invivo biomechanical study. J Biomech 2005; 38: 503
Akali A et al. Decrease in adhesion formation by a single application of 5-fluorouracil after flexor tendon injury. Plast Reconstr Surg 1999; 103: 151-8
Beredjiklian P K. Biologic aspects of flexor tendon laceration and epair. J Bone Joint Surg Am. 2003; 85A (3): 539-50
Berglund M, et al. Patterns of mRNA expression for matrix molecules and growth factors in flexor tendon injury: Differences in the regulation between tendon and tendon sheath. J Hand Surg 2006; 31A: 1279-1287
Branford O, et al. A novel biometric material to engineer post-surgical adhesion using the injured digital flexor tendon—synovial complex as an in vivo model. Plast Reconstr Surg 2008; 121: 781-793
Chang J, et al. Studies in flexor tendon wound healing: neutralizing antibody toe TGF-β1 increases postoperative range of motion. Plast Reconstr Surg 2000; 105: 148-155
Fu S-C, et al. TGF-β1 reverses the effects of matrix anchorage on the gene expression of decorin and procollagen type I in tendon fibroblasts. Clin Orthopaed Rel Res 2005; 431: 226-232
Gelberman R H, Manske P R. Factors influencing flexor tendon adhesions. Hand Clin 1985a; 1: 35-42
Gelberman R H et al, The Early stages of flexor tendon healing: a morphologic study of the first fourteen days. J Hand Surg (Am) 1985b; 10: 776-784
Gelberman R H et al., Flexor tendon repair. J Orthop Res 1986, 4: 119-28
Gelberman et al. Fibroblast chemotaxis after tendon repair. J Hand Surg 1991; 16: 686-93
Golash A et al. Efficacy of ADCON-T/N after primary flexor tendon repair in zone II: A controlled clinical trial. J Hand Surg 2003; 28B (2): 113-115
Hatano et al, Adhesions from flexor tendon surgery: An animal study comparing surgical techniques. J Hand Surg (Am) 2000; 25: 252-259
He Q, et al., repair of flexor tendon defects of rabbit with tissue engineering method. Chin J Traumatol 2002; 5: 200-8
Jones M E, al. The role of human-derived fibrin sealant in the reduction of postoperative flexor tendon adhesion formation in rabbits. J Hand Surg (Br) 2002; 27: 278-82.
Kakar S, et al. Differential cellular response within the rabbit tendon unit following tendon injury. J Hand Surg [Br] 1998; 23: 627-632
Khan U et al. Patterns of cellular activation after tendon injury. J Hand Surg 1996; 21B 813-820
Khan U, et al., Differences in proliferative rate and collagen lattice contraction between endotenon and synovial fibroblasts. J Hand Surg 1998; 23A: 266-273
Khan U, et al. Modulation of the formation of adhesions during the healing of injured tendons. J Bone Joint Surg Br. 2000; 82: 1054-8
Manske P R. Flexor Tendon Healing. J Hand Surgery 1988; 13: 237-45.
Manske P R et al, Flexor tendon healing. Hand Clin 1985; 1: 25-34
Matthews P, Richards H. Factors in the adherence of flexor tendon after repair: an experimental study in the rabbit. J Bone Joint Surg 1976; 58B: 230-236
Mills P C, et al., Surface-active phospholipids (surfactant) in equine tendon and tendon sheath fluid. NZ Vet J 2005; 53:154-6
Moro-oka T et al. Mixture of hyaluronic acid and phospholipd prevents adhesion formation on the injured flexor tendon in rabbits. J Orthop Res 2000; 18: 835-40
Morgan L W, et al. Glucose degradation products (GDP) retard re-mesothelialisation independently of D-glucose concentration. Kidney Int 2003; 64: 1854-1866
Momose T, Amadio P C, Zhao C, et al. Suture techniques with high breaking strength and low gliding resistance. Acta Orthop Scand 2001; 72: 635-641.
Ngo M., et al. Differential expression of transforming-growth factor beta receptors in a rabbit zone II tendon wound healing model. Plast Reconstr Surg 2001; 108: 1260-7
Nyska M, et al. Decreased adhesion formation in flexor tendons by topical application of enriched collagen solution—a histological study. Arch Orthop Trauma Surg 1987:106:192-4
Pennisi E., Tending tender tendons. Science 2002, 295: 1011
Pfaffl M W, Horgan G W, Dempfle L. Relative expression software tool (REST) for group-wise comparison and statistical analysis of relative expression results in real-time PCR. Nucleic Acids Res 2002: 30: e36.
Porat S et al. Improvement of gliding function of flexor tendons by topically applied enriched collagen solution. J Bone Joint Surg Br 1980 62B (2): 208-13
Potenza A D. Critical evaluation of flexor tendon healing and adhesion formation within artificial digital sheaths. J Bone and Joint Surg 1963; 45A: 1217-1233
Ragoowansi R, et al. differences in morphology, cytoskeletal architecture and proteas production between zone II tendon and synovial fibroblasts in vitro. J Hand Surg (Br) 2003; 28: 465-470
Rolfe K J et al. A role for TGF-β1 induced cellular responses during the wound healing of the non-scarring human fetus? J Invest Dermatol 2007; 127:2656-2667
Speer D P, Feldman S, Chvapil M. The control of periteninous adhesions using topical beta-aminopropionitrile base. J Surg Res 1985; 38: 252-257
Strickland J W. Development of flexor tendon surgery: twenty-five years of progress. J Hand Surg [Am] 2000; 25: 214-35
Thomas S C et al, Hyaluronic acid and its effect on postoperative adhesions in the rabbit flexor tendon. A preliminary look. Clin Orthop 1986; 206: 281-289
Williams I F et al, The distribution of types I and III collagen and fibronectin in the healing equine tendon. Connect Tissue Res 1984; 12: 211-227
Zhang A Y, et al. Inhibition of TGF-β-induced collagen production in rabbit flexor tendons. J Hand Surg 2004; 29A: 2300

1. A phospholipid in a crystalline form.

2. A phospholipid as defined in claim 1 which has been freeze dried.

3. A phospholipid as defined in claim 1 which is sterilised.

4. A phospholipid as defined in claim 1 which is in a form such that when dispersed at room temperature in a polar solvent, the dispersion formed comprises discrete crystals; preferably the phospholipid is in a form such that when dispersed at 37° C. in a polar solvent, the dispersion formed comprises a fine micellar dispersion which has substantially no visible agglomerations of micelles; more preferably the polar solvent is an isotonic polar solvent.

5. A phospholipid as defined in claim 1 which is in the form of a respirable particle.

6. A phospholipid as defined in claim 1 which is in the form of a micronized particle.

7. A phospholipid as defined in claim 1 which has the following general formula: 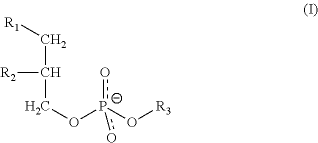
wherein R₁ and R₂ each independently represents a hydrogen atom or a fatty acid acyl residue, and R₃ represents a hydrogen atom or a choline, glycerol, ethanolamine, serine or inositol group wherein R₁ and R₂ cannot both represent a hydrogen atom.

8. A phospholipid as defined in claim 7 wherein the fatty acid acyl residue independently represented by R₁ or R₂ is palmitoyl C16:0, stearoyl C18:0, oleoyl C18:1 and/or oleoyl C18:2.

9. A phospholipid in a crystalline form wherein the phospholipid is sterilized and has the following general formula: 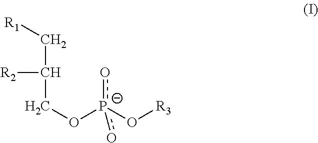
wherein R₁ and R₉ each independently represents a hydrogen atom or a fatty acid acyl residue, and R₃ represents a hydrogen atom or a choline, glycerol, ethanolamine, serine or inositol group wherein R₁ and R₂ cannot both represent a hydrogen atom.

10. A process of preparing a phospholipid which method comprises;
dispersing a phospholipid in a polar solvent;
homogenising the phospholipid dispersion; and either
(a) filtering the homogenised phospholipid to sterilise it; and freeze drying the filtered phospholipid dispersion; or
(b) freeze drying the phospholipid dispersion; and micronising the freeze dried phospholipid.

11. A process as defined in claim 10 wherein the filtering step uses a 0.5 μm filter.

12. A phospholipid obtainable by the process as defined in claim 10.

13. A pharmaceutical composition which comprises a phospholipid as defined in claim 1 in association with a pharmaceutically acceptable diluent.

14. A pharmaceutical composition as defined in claim 13 wherein the only active ingredient is the phospholipid.

15. A kit for preparing a pharmaceutical composition as defined in claim 13 which kit has at least two parts wherein a first part comprises a phospholipid and a second part comprises a pharmaceutically acceptable diluent.

16. (canceled)

17. (canceled)

18. A method of treating damaged tissue which method comprises applying to a human or animal patient in need of such treatment a therapeutically effective amount of a phospholipid as defined in claim 1.

19. A method of treating damaged tissue which method comprises applying to a human or animal patient in need of such treatment a therapeutically effective amount of a pharmaceutical composition as defined in claim 13.

20. A method of treating damaged tissue which method comprises applying to a human or animal patient in need of such treatment a therapeutically effective amount of a kit as defined in claim 16.


SEQUENCE LISTING (14 sequences)

SEQ ID NO: 1 - DNA, 19 bp, Artificial Sequence
  Primer
caaacctcttcctgaagcc

SEQ ID NO: 2 - DNA, 19 bp, Artificial Sequence
  Primer
attatagcaccattgagac

SEQ ID NO: 3 - DNA, 28 bp, Artificial Sequence
  Primer
gaggctcacgtccggctgtacccctcca

SEQ ID NO: 4 - DNA, 28 bp, Artificial Sequence
  Primer
tccttctgcccacagcccagcccccagc

SEQ ID NO: 5 - DNA, 21 bp, Artificial Sequence
  Primer
ttcttggtgctcctggcattc

SEQ ID NO: 6 - DNA, 23 bp, Artificial Sequence
  Primer
gcaatccgttgtgtccctttatg

SEQ ID NO: 7 - DNA, 16 bp, Artificial Sequence
  Primer
ggatttggccgcattg

SEQ ID NO: 8 - DNA, 24 bp, Artificial Sequence
  Primer
caacatccactttgccagagttaa

SEQ ID NO: 9 - DNA, 24 bp, Artificial Sequence
  Primer
tcgggaggaagaagacagatgagc

SEQ ID NO: 10 - DNA, 22 bp, Artificial Sequence
  Primer
accactgccaaagcctaagcac

SEQ ID NO: 11 - DNA, 17 bp, Artificial Sequence
  Primer
ggatttggccgcattgg

SEQ ID NO: 12 - DNA, 24 bp, Artificial Sequence
  Primer
caacatccactttgccagagttaa

SEQ ID NO: 13 - DNA, 21 bp, Artificial Sequence
  Primer
gctcgtcgtcgacaacggctg

SEQ ID NO: 14 - DNA, 25 bp, Artificial Sequence
  Primer
caaacatgatctgggtcatcttctc